SEQUENCE LISTING (83 sequences)

SEQ ID NO: 1 - DNA, 1386 bp, Sphingopyxis sp. C-1
atgaaaaacctgatcctggcgaccgacagctacaagcagagccactttctgcaatatccgcccgaggcgcgcgtaatcagcgcctatgtcgaggcgcggccaaaccccttttccgaagagattgtctttctgggtctccagccgctgctggtcgactatttcagccagccgatcaacgcggcggatatcgacgaggccgaggcgatctgcatcgcgcacggcgttccgttcaaccgtgcggggtgggaggcgatcgtcgccgatcatggcggttatctgccgctggagatcaaggcgctgcccgaaggcgcgatcgtgcccgcgggcgtgccgctggtacagctcgaaaataccgacccgcgcatgccctggctgacgaccttcatagagacggcaatgctgcgtgcgatctggtatccgacgacggtcgcgacgctgagctggaagtgcaaacaggtgatccgcgcggggctcgaaaagacgtccgacgatgtggagggccagcttcccttcaagctccacgatttcggcgcgcgcggggtttcttccgccgaaagtgcgggtctgggtgggctcgcacatctcgtcaatttccagggcaccgacacgatggaggcgctggtcgcggcgcggcgctactatggcgccgacatggcgggcttttctattcccgccgccgagcacagtacaatgacgagctggggccgcgaccgtgaagaggatgcctatcgcaacatgctcgaccggttcgaaggcgagggacgcatcgtcgcggtcgtctccgacagctatgatctcgatacggcggtcaccgacatctggggcggcagcttgcgcgagaaggtgctggggcgcgcgggcacgctggtcgtacggcccgacagcggcgatccgatcgaaacgccgctgcgcacggtgaaaacgctgtgggaaaagtttggcggccatgtgaacggcaagggctatcgcgtcctcgatccgcatgtccgcgtgatccagggcgacggaatgaccgtcgacagcatcggccggcttgttcagcggatgatcgaggaaggtttcgcgatcgacaatatcgctttcggcatgggcggcgggatgctgcagcacgtcaaccgcgacacgctgcgcttcgcgatgaaggcgaatgcgatgctgggcagcgacggcgtgtggcacgatgtcttcaagatgccgagtaccgatccgggcaaggcgagcaaggccggacggcaggccgtcgtgctgaaggacggccggatggccgcagcgcggctcgacagcgtcgcggtgggcgaagatctgctggtgccggtatggcggaatggcgaactactcgtccgccacgacttcgacgcggtgcggaagcggtccgaaggcaggtga

SEQ ID NO: 2 - DNA, 1386 bp, Artificial Sequence
  Synthetic sequence, Nicotinamide phosphoribosyltransferase SSC (optimized)
atgaagaatctgattctggccaccgatagctataaacagagccattttctgcagtatccgcctgaagcacgtgttattagcgcatatgttgaagcccgtccgaatccgtttagcgaagaaattgtttttctgggtctgcagccgctgctggttgattatttcagccagccgattaatgcagccgatattgatgaagcagaagccatttgtattgcacatggtgttccgtttaatcgtgcaggttgggaagcaattgttgcagatcatggtggttatctgccgctggaaattaaagcactgccggaaggtgcaattgttccggcaggcgttccgctggttcagctggaaaataccgatccgcgtatgccgtggctgaccacctttattgaaaccgcaatgctgcgtgcaatttggtatccgaccaccgttgcaaccctgagctggaaatgcaaacaggttattcgtgccggtctggaaaaaaccagtgatgatgttgaaggtcagctgccgtttaaactgcatgattttggtgcacgtggtgttagcagcgcagaaagcgcaggtttaggtggtctggcacatctggttaattttcagggcaccgataccatggaagcactggttgcagcccgtcgttattatggtgcagatatggcaggttttagcattccggcagcagaacatagcaccatgaccagctggggtcgtgatcgtgaagaagatgcatatcgtaatatgctggatcgctttgaaggtgaaggtcgtattgttgccgttgttagcgatagttatgatctggataccgcagttaccgatatttggggtggtagcctgcgtgaaaaagttctgggtcgtgcgggtacactggttgttcgtccggatagcggtgatccgattgaaacaccgctgcgtaccgttaaaaccctgtgggaaaaatttggtggtcatgtgaatggtaaaggttatcgtgttctggatccgcatgttcgtgttattcaaggtgatggtatgaccgttgatagcattggtcgtctggtgcagcgtatgattgaagaaggttttgccattgataacattgcctttggtatgggtggtggtatgctgcaacatgttaatcgtgataccctgcgttttgcaatgaaagcaaatgcaatgctgggtagtgatggtgtttggcatgatgtgtttaaaatgccgagcaccgatccgggtaaagcaagcaaagcaggtcgtcaggcagttgttctgaaagatggtcgtatggcagcagcacgtctggatagcgttgcagttggtgaagatctgctggttccggtttggcgtaatggtgaactgctggtgcgtcacgattttgatgcagttcgtaaacgtagcgaaggtcgttaa

SEQ ID NO: 3 - protein, 461 aa, Sphingopyxis sp. C-1
MKNLILATDSYKQSHFLQYPPEARVISAYVEARPNPFSEEIVFLGLQPLLVDYFSQPINAADIDEAEAICIAHGVPFNRAGWEAIVADHGGYLPLEIKALPEGAIVPAGVPLVQLENTDPRMPWLTTFIETAMLRAIWYPTTVATLSWKCKQVIRAGLEKTSDDVEGQLPFKLHDFGARGVSSAESAGLGGLAHLVNFQGTDTMEALVAARRYYGADMAGFSIPAAEHSTMTSWGRDREEDAYRNMLDRFEGEGRIVAVVSDSYDLDTAVTDIWGGSLREKVLGRAGTLVVRPDSGDPIETPLRTVKTLWEKFGGHVNGKGYRVLDPHVRVIQGDGMTVDSIGRLVQRMIEEGFAIDNIAFGMGGGMLQHVNRDTLRFAMKANAMLGSDGVWHDVFKMPSTDPGKASKAGRQAVVLKDGRMAAARLDSVAVGEDLLVPVWRNGELLVRHDFDAVRKRSEGR

SEQ ID NO: 4 - DNA, 1416 bp, Chitinophaga pinensis
atgacaaaggaaaacctcattttgttagctgatgcctacaaatactcccaccacaaactttacatccccggtacagaatatatctattcttatttcgaaagcagaggcggtaaattcaatgaaaccgtcttctacggactccagtatttcctgatggaatacctccagggtgctgttatcaccaaagaaaaacttgacgaagcagaagctaccttgctggaagtcttcggtcgtaacgatgtattcgaccgcacccgcttcgaatatatcatcgagaaacacggaggccgcctccctgtacgtatcaaagcagtaccggaaggaacagtaaccggcgtacgtaacgtgctgatgaccattgaaaatacagatcctaattgttactgggtgaccaacttcctggaaacgctcctgatgcagatatggtatccatgtacagtagccacgctttccagagagatcaaaaaaactgttaaacaatactataacgagacggccagtgaagcggctttcgcaggaattgatttcgtactgaacgacttcggtttccgtggcgccagctctgtagaaagtgcaggtataggcggtagcgctcacctgatcaacttctccggtagcgataccctgatcggttccacttttgccaaacgttattaccaggcgccggtagctcccggtctctctatccccgctacagaacactctatcgtgactatgctgggtgaagaaggagaactggaaatcttccgtcacatactgaatgcgttccctaccggtactatcgcctgtgtatctgactcttacaatatcctccgtgcctgccgcgaatactggggtactgaactgaaagaacagatcctcagcagacaaggtaccctggtgatccgtcccgacagcggtgacgccattcagaccctactgaaagtatttgaaattctgatggaaaccttcggttataccgtcaatgaaaaaggctataaagtattacctccacaggtgagagtgatccagggtgatggtattagctattcttccattcctccgatcttcgaagctcttaaacaggccggtatcagcgctgaaaacctggtgctgggtatgggaggagccttgctgcaacgtgtaaacagagatacacaggaatacgccctgaaatgctcttttgcacaggtgaacggtaaagccatcaacgtacagaaaaacccgctggaactggatgcgaacggtaatacccgtgtatccttcaagaagtctaaatccggtaaacagaaactggtagtagaaaacggtatttatacttctctacctgaaaatgaagcacctgcactggctgaccagctggtaactgtcttcgaagacggagagatcaaaaaagcatactcttttgaacagatcagaaagaacgcaactatttaa

SEQ ID NO: 5 - DNA, 1416 bp, Artificial Sequence
  Synthetic sequence, Nicotinamide phosphoribosyltransferase CP (optimized)
atgaccaaagaaaacctgattctgctggcagatgcatacaaatatagccaccacaaactgtatattccgggaaccgaatatatctacagctattttgaaagccgtggtggcaaatttaacgaaaccgttttttatggcctgcagtacttcctgatggaatatctgcagggtgcagttatcacaaaagaaaaactggatgaagcagaagcaaccctgctggaagtttttggtcgtaatgatgtttttgatcgcacccgctttgaatacatcattgaaaaacatggtggtcgtctgccggttcgtattaaagcagttccggaaggcaccgttaccggtgttcgtaatgttctgatgaccattgaaaataccgatccgaattgttattgggtgaccaattttctggaaacactgctgatgcagatttggtatccgtgtaccgttgcaaccctgagccgtgaaatcaaaaaaaccgttaaacagtattacaatgaaaccgcaagcgaagcagcatttgcaggtattgattttgtgctgaacgattttggttttcgtggtgcaagcagcgttgaaagtgccggtattggtggtagcgcacatctgattaactttagcggtagcgataccctgattggtagcacctttgcaaaacgttattatcaggcaccggttgcaccgggtctgagcattccggcaacagaacattcaattgttaccatgctgggtgaagaaggtgaactggaaatttttcgccatattctgaatgcatttccgaccggcaccattgcatgtgttagcgatagctataacattctgcgtgcatgtcgtgaatattggggcaccgaactgaaagagcagattctgagccgtcagggcaccctggttattcgtccggatagcggtgatgcaattcagacgctgctgaaagtgtttgaaattctgatggaaacctttggctataccgtgaacgaaaaaggctataaagttctgcctccgcaggttcgtgttattcaaggtgatggtattagctatagcagcattccgcctatttttgaagcactgaaacaggcaggtattagcgcagaaaatctggttttaggtatgggtggtgcactgctgcagcgtgttaatcgtgatacccaagaatatgcactgaaatgtagctttgcacaggttaatggcaaagccattaatgtgcagaaaaatccgctggaactggatgcaaatggtaatacccgtgtgagtttcaaaaaaagcaaaagcggtaaacagaaactggtggtggaaaatggtatttataccagcctgccggaaaatgaagcaccggcactggcagatcagctggttaccgtttttgaagatggcgaaattaagaaagcctatagctttgagcagatccgtaaaaacgcaaccatctaa

SEQ ID NO: 6 - protein, 471 aa, Chitinophaga pinensis
MTKENLILLADAYKYSHHKLYIPGTEYIYSYFESRGGKFNETVFYGLQYFLMEYLQGAVITKEKLDEAEATLLEVFGRNDVFDRTRFEYIIEKHGGRLPVRIKAVPEGTVTGVRNVLMTIENTDPNCYWVTNFLETLLMQIWYPCTVATLSREIKKTVKQYYNETASEAAFAGIDFVLNDFGFRGASSVESAGIGGSAHLINFSGSDTLIGSTFAKRYYQAPVAPGLSIPATEHSIVTMLGEEGELEIFRHILNAFPTGTIACVSDSYNILRACREYWGTELKEQILSRQGTLVIRPDSGDAIQTLLKVFEILMETFGYTVNEKGYKVLPPQVRVIQGDGISYSSIPPIFEALKQAGISAENLVLGMGGALLQRVNRDTQEYALKCSFAQVNGKAINVQKNPLELDANGNTRVSFKKSKSGKQKLVVENGIYTSLPENEAPALADQLVTVFEDGEIKKAYSFEQIRKNATI

SEQ ID NO: 7 - DNA, 1425 bp, Burkholderia cenocepacia
atgcccgccgccaccgctcccgcctccgccgccgcccggctcgaacgcctgccgttctccggctatcacaagcgcatcttcttcatcatcgcgatcgcgttcttcttcgattcggtcgacctcggcacgatgacgttcgtgctcggctcgattcgcaaggagttcgggctgtcgaccgcggccgccggcctcgtcgcgagcgcgagcttcttcgggatggtgctcggcgcggccgtcgccggcctgctcgccgaccgtttcggccgtcggccggtgttccagtggagcatggtgctgtggggcgccgcgtcgtacctgtgctcgaccgcgcagagcgtcgacgcgttgatcgtctatcgcgtgttgctcggcatcgggatggggatggagtttccggtcgcgcagacgctgctgtccgaattcgtgccgaccgagaaacgcggccgcctgatcgcgctgatggacggcttctggccgctcggcttcatcacggccggcatcgtcgcgtatttcgtgctgccgcagttcggctggcgcaccgtgttcgcgctgctcgcgattccggccgtgttcgtgctcgtcgtacgccgcatcgtgccggaatcgccgcgctggctcgaacatgcgggccggcacgcggaagccgacacggtgatgcacacgatcgaggcgaaggtgatgcgctcggccggcgtcacgacgctgccgccgccgtcgcggctcgccgagccggccgccgcacgcggtcgcggcgcgctgcgcgagatctggagcggcgtgtaccgtcgccgcacggtgatggtgtggctgctgtggttcttcgcgctgctcggcttctacggcctcacgtcgtggctcggcgcgctgctgcagcaggccggcttcgaagtcacgaaatcggtgttctacacggtgctgatctcgctcggcggcgtgccgggcttcctgtgcgccgcgtggctcgtcgaacgctggggccgcaagccgacctgcatcgcatcgctgatcggcggcggtgcgatggcgtacgcatacggccagagcgcgctgtacggcggcagcacgacgctgctgatcgtcacgggcctcgcgatgcagttcttcctgttcgggatgtgggcggcgctgtacacgtacacgcccgagctgtacggcaccggcgcacgcgcgaccggttcgggcttcgcgtcggcgatcggtcgcgtcggttcgctgatcgggccttacgtggtcggcgtcgtgttgccggtgttcggccagggcggcgtgttcacgctcggcgcgctgtcgttcgtcgcggcggccatcgccgtgtggacactgggaatcgagacgaagggcctcgcgctggagcaactggcggcaggcgacgacgcgggcggcaacggccggtatccggcgacggcggcggacaaggcgtcctga

SEQ ID NO: 8 - DNA, 1425 bp, Artificial Sequence
  Synthetic sequence, Niacin transporter niaP (optimized)
atgcctgcagcaaccgcaccggcaagcgcagcagcacgtctggaacgtctgccgtttagcggttatcataaacgcatctttttcattatcgcgatcgccttttttttcgatagcgttgatctgggcaccatgacctttgttctgggtagcattcgtaaagaatttggtctgagcaccgcagccgcaggtctggttgcaagcgcaagcttttttggtatggttctgggtgcagcagttgcaggtctgctggcagatcgttttggtcgtcgtccggtttttcagtggtcaatggttctgtggggtgcagccagctatctgtgtagcaccgcacagagcgttgatgcactgattgtttatcgtgttctgttaggtattggtatgggtatggaatttccggttgcacagaccctgctgagcgaatttgttccgaccgaaaaacgtggtcgtctgattgcactgatggatggtttttggcctctgggttttattaccgcaggtattgttgcatatttcgttctgccgcagtttggttggcgtaccgtttttgcactgctggcaattccggcagtttttgtgctggttgttcgtcgtattgttccggaaagtccgcgttggctggaacatgcaggtcgtcatgccgaagcagataccgttatgcataccattgaagcaaaagttatgcgtagtgccggtgttacaaccctgcctccgcctagccgtctggcagaacctgcagcagcccgtggtcgtggtgcactgcgtgaaatttggagcggtgtgtatcgtcgtcgtaccgtgatggtttggctgctgtggtttttcgccctgctgggcttctatggtctgaccagctggctgggtgccctgctgcaacaggcaggttttgaagttaccaaaagcgtgttttataccgtcctgattagcttaggtggtgttccgggttttctgtgtgcagcctggctggttgaacgttggggtcgtaaaccgacctgtattgcaagcctgattggtggtggtgcaatggcctatgcatatggtcagagcgcactgtatggtggtagcaccacactgctgattgttaccggtctggcaatgcagttttttctgtttggtatgtgggcagccctgtatacctatacaccggaactgtatggcacaggtgcacgtgccaccggtagcggttttgccagcgcaattggtcgtgttggttcactgattggtccgtatgttgttggtgttgttctgccggtttttggtcaaggtggtgtgtttaccctgggtgcactgagctttgttgcagccgcaattgcagtttggaccctgggtattgaaaccaaaggtctggcactggaacagctggcagccggtgatgatgccggtggtaatggtcgttatccggcaacagcagcagataaagcaagctaa

SEQ ID NO: 9 - protein, 474 aa, Burkholderia cenocepacia
MPAATAPASAAARLERLPFSGYHKRIFFIIAIAFFFDSVDLGTMTFVLGSIRKEFGLSTAAAGLVASASFFGMVLGAAVAGLLADRFGRRPVFQWSMVLWGAASYLCSTAQSVDALIVYRVLLGIGMGMEFPVAQTLLSEFVPTEKRGRLIALMDGFWPLGFITAGIVAYFVLPQFGWRTVFALLAIPAVFVLVVRRIVPESPRWLEHAGRHAEADTVMHTIEAKVMRSAGVTTLPPPSRLAEPAAARGRGALREIWSGVYRRRTVMVWLLWFFALLGFYGLTSWLGALLQQAGFEVTKSVFYTVLISLGGVPGFLCAAWLVERWGRKPTCIASLIGGGAMAYAYGQSALYGGSTTLLIVTGLAMQFFLFGMWAALYTYTPELYGTGARATGSGFASAIGRVGSLIGPYVVGVVLPVFGQGGVFTLGALSFVAAAIAVWTLGIETKGLALEQLAAGDDAGGNGRYPATAADKAS

SEQ ID NO: 10 - DNA, 582 bp, Streptococcus pneumoniae TIGR4
ttgtctggtttattgtaccatactagtgtatatgcagttaaaaaggagattcttgtgaatacacggaaaaagacacaatttatgacaatgacagcccttttaacggctattgcgattttgattccaattgttatgcctttcaagattgtcattccacctgcttcctatactttggggagccacatcgctatttttatagccatgttcttgtcgcccttgatggcagtttttgtcatcctagcctctagttttggatttttgatggctggctatcccatggttatcgtttttcgggctttttcccatatatcttttggtgctttaggagctctttacctacaaaaattccccgataccctagataaaccaaaatcttcctggattttcaactttgttttggctgttgttcatgcccttgctgaagtattggcctgtgtcgttttttacgcaacttctggtaccaatgtagaaaatatgttttatgttctatttgtactagttggatttggtacaattatccatagtatggtagactatacattagcactagctgtctataaagtgcttcgaaaacgccgttaa

SEQ ID NO: 11 - DNA, 582 bp, Artificial Sequence
  Synthetic sequence, Niacin transporter niaX (optimized)
ttgagcggtctgctgtatcacaccagcgtttatgcagtgaaaaaagaaattctggtgaacacccgtaaaaaaacccagtttatgaccatgaccgcactgctgaccgcaattgccattctgattccgattgttatgccgttcaaaattgttattccgcctgcaagctataccctgggtagccatattgcaatctttattgcaatgtttctgagtccgctgatggccgtttttgttattctggcaagcagctttggttttctgatggcaggttatccgatggttattgtttttcgtgcatttagccacattagctttggtgcactgggtgccctgtatctgcagaaatttccggatacactggataaaccgaaaagcagctggatctttaactttgttctggcagttgttcatgcactggccgaagttctggcatgtgttgttttttatgcaaccagcggcaccaatgtggaaaatatgttttatgttctgttcgtgctggttggctttggcaccattattcatagcatggttgattatacactggccctggcagtttataaagttctgcgtaaacgtcgctaa

SEQ ID NO: 12 - protein, 193 aa, Streptococcus pneumoniae TIGR4
LSGLLYHTSVYAVKKEILVNTRKKTQFMTMTALLTAIAILIPIVMPFKIVIPPASYTLGSHIAIFIAMFLSPLMAVFVILASSFGFLMAGYPMVIVFRAFSHISFGALGALYLQKFPDTLDKPKSSWIFNFVLAVVHALAEVLACVVFYATSGTNVENMFYVLFVLVGFGTIIHSMVDYTLALAVYKVLRKRR

SEQ ID NO: 13 - DNA, 651 bp, Bacillus mycoides
atggttagaagtccactttttttactcatttctagtattgtttgcatattagttggattctatatccgatcaagttatattgaaattttcgcatcggttatggggattattaatgtttggttacttgcaagggaaaaggtatcaaactttttatttgggatgattacagttgcggtatttctgtatattttcactacacaaggcttatacgcaatggcagtattagcagccttccaatttatatttaatgtgtacggttggtattactggattgcgcgtagtggggaggagaaggtaaaaccgacagttcgcttagatttgaaaggatggattatttatatactttttattttagttgcttggattggttggggatattatcaagtccgttatttagaatcgacaagtccatatttagatgctttaaacgctgtattaggattagtagctcaatttatgctgagccgaaaaatcttagaaaattggcatttatggattttgtataacatagttagcattgtgatttatatttcaacgggcttatacgtcatgttagtattagctattattaatctatttttatgtatcgatggattgctagaatggaagaaaaaccataaagagcgagaacgtgtaaataattatatttag

SEQ ID NO: 14 - DNA, 651 bp, Artificial Sequence
  Synthetic sequence, Nicotinamide mononucleotide transporter pnuC (optimized)
atggttcgtagtccgctgtttctgctgattagcagcattgtttgtattctggtgggcttttatatccgcagcagctatattgaaattttcgcaagcgttatgggcatcattaatgtttggctgctggcacgtgaaaaagtgagcaattttctgtttggtatgattaccgttgccgtgttcctgtatatctttaccacacagggtctgtatgcaatggcagttctggcagcatttcagtttatctttaatgtgtatggctggtattattggattgcacgtagcggtgaagaaaaagttaaaccgaccgttcgtctggatctgaaaggttggattatctatatcctgtttattctggttgcctggattggttggggttattatcaggttcgttatctggaaagcaccagtccgtatctggatgcactgaatgcagttttaggtctggttgcacagtttatgctgagccgtaaaattctggaaaattggcatctgtggatcctgtataatatcgtgagcatcgtgatttatatcagcactggcctgtatgttatgctggttctggccattattaacctgtttctgtgtattgatggtctgctggaatggaaaaagaaccataaagaacgtgaacgcgtgaacaactacatctaa

SEQ ID NO: 15 - protein, 216 aa, Bacillus mycoides
MVRSPLFLLISSIICILVGFYIRSSYIEIFASVMGIINVWLLAREKVSNFLFGMITVAVFLYIFTTQGLYAMAVLAAFQFIFNVYGWYYWIARSGEEKVKPTVRLDLKGWIIYILFILVAWIGWGYYQVRYLESTNPYLDALNAVLGLVAQFMLSRKILENWHLWILYNIVSIVIYISTGLYVMLVLAIINLFLCIDGLLEWKKNHKERERVNNYI

SEQ ID NO: 16 - DNA, 1650 bp, Escherichia coli
atgaaaaacatcaatccaacgcagaccgctgcctggcaggcactacagaaacacttcgatgaaatgaaagacgttacgatcgccgatctttttgctaaagacggcgatcgtttttctaagttctccgcaaccttcgacgatcagatgctggtggattactccaaaaaccgcatcactgaagagacgctggcgaaattacaggatctggcgaaagagtgcgatctggcgggcgcgattaagtcgatgttctctggcgagaagatcaaccgcactgaaaaccgcgccgtgctgcacgtagcgctgcgtaaccgtagcaataccccgattttggttgatggcaaagacgtaatgccggaagtcaacgcggtgctggagaagatgaaaaccttctcagaagcgattatttccggtgagtggaaaggttataccggcaaagcaatcactgacgtagtgaacatcgggatcggcggttctgacctcggcccatacatggtgaccgaagctctgcgtccgtacaaaaaccacctgaacatgcactttgtttctaacgtcgatgggactcacatcgcggaagtgctgaaaaaagtaaacccggaaaccacgctgttcttggtagcatctaaaaccttcaccactcaggaaactatgaccaacgcccatagcgcgcgtgactggttcctgaaagcggcaggtgatgaaaaacacgttgcaaaacactttgcggcgctttccaccaatgccaaagccgttggcgagtttggtattgatactgccaacatgttcgagttctgggactgggttggcggccgttactctttgtggtcagcgattggcctgtcgattgttctctccatcggctttgataacttcgttgaactgctttccggcgcacacgcgatggacaagcatttctccaccacgcctgccgagaaaaacctgcctgtactgctggcgctgattggcatctggtacaacaatttctttggtgcggaaactgaagcgattctgccgtatgaccagtatatgcaccgtttcgcggcgtacttccagcagggcaatatggagtccaacggtaagtatgttgaccgtaacggtaacgttgtggattaccagactggcccgattatctggggtgaaccaggcactaacggtcagcacgcgttctaccagctgatccaccagggaaccaaaatggtaccgtgcgatttcatcgctccggctatcacccataacccgctctctgatcatcaccagaaactgctgtctaacttcttcgcccagaccgaagcgctggcgtttggtaaatcccgcgaagtggttgagcaggaatatcgtgatcagggtaaagatccggcaacgcttgactacgtggtgccgttcaaagtattcgaaggtaaccgcccgaccaactccatcctgctgcgtgaaatcactccgttcagcctgggtgcgttgattgcgctgtatgagcacaaaatctttactcagggcgtgatcctgaacatcttcaccttcgaccagtggggcgtggaactgggtaaacagctggcgaaccgtattctgccagagctgaaagatgataaagaaatcagcagccacgatagctcgaccaatggtctgattaaccgctataaagcgtggcgcggttaa

SEQ ID NO: 17 - DNA, 1650 bp, Artificial Sequence
  Synthetic sequence, Phosphoglucose isomerase pgi (optimized)
atgaagaacattaatccgacacagaccgcagcatggcaggcactgcagaaacattttgatgaaatgaaagatgtgaccattgcagacctgtttgcaaaagatggtgatcgctttagcaaatttagcgccacctttgatgatcagatgctggttgattatagcaaaaaccgcattaccgaagaaaccctggcaaaactgcaggatctggcaaaagaatgtgatctggcaggcgcaattaaaagcatgtttagcggtgaaaaaatcaaccgtaccgaaaatcgtgcagttctgcatgttgcactgcgtaatcgtagcaataccccgattctggttgatggtaaagatgttatgccggaagttaatgccgttctggaaaaaatgaaaacctttagcgaagccattatcagcggtgaatggaaaggttataccggtaaagcaattaccgatgtggtgaatattggtattggtggtagcgatctgggtccgtatatggttaccgaagcactgcgtccgtataaaaaccatctgaatatgcattttgtgagcaatgttgatggcacccatattgcagaagtgctgaaaaaagttaatccggaaaccacactgtttctggttgcaagcaaaacatttaccacacaagaaaccatgaccaatgcacatagcgcacgtgattggtttctgaaagcagccggtgatgaaaaacatgtggcaaaacactttgcagcactgagcaccaatgcaaaagcagtgggtgaatttggcattgataccgccaatatgtttgaattctgggattgggttggtggtcgttatagcctgtggtcagcaattggtctgagcattgttctgagtattggctttgataactttgtggaactgctgagcggtgcacatgcaatggataaacattttagcaccacaccggcagaaaaaaatctgccggttctgctggcactgattggtatttggtataacaacttttttggtgccgaaaccgaagcaattctgccgtatgatcagtatatgcatcgttttgcagcatattttcagcagggtaatatggaaagcaacggcaaatatgttgatcgcaatggtaatgtggtggattatcagaccggtccgattatttggggtgaaccgggtacaaatggtcagcatgcattttatcaactgattcatcagggtacaaaaatggtgccgtgtgattttattgcaccggcaattacccataatccgctgagcgatcatcatcagaaactgctgtcaaatttctttgcccagaccgaagcgctggcatttggtaaaagccgtgaagttgttgaacaagaatatcgcgatcagggtaaagatccggcaacactggattatgttgttccgtttaaagtgtttgaaggtaatcgtccgaccaatagcattctgctgcgtgaaattaccccgtttagcctgggtgccctgattgcactgtatgaacacaaaattttcacccagggtgtgatcctgaacatttttacctttgatcagtggggtgttgaactgggtaaacagctggcaaatcgtattctgccggaactgaaagatgataaagaaatcagcagccatgatagcagtaccaatggtctgattaatcgttataaagcctggcgtggttaa

SEQ ID NO: 18 - protein, 549 aa, Escherichia coli
MKNINPTQTAAWQALQKHFDEMKDVTIADLFAKDGDRFSKFSATFDDQMLVDYSKNRITEETLAKLQDLAKECDLAGAIKSMFSGEKINRTENRAVLHVALRNRSNTPILVDGKDVMPEVNAVLEKMKTFSEAIISGEWKGYTGKAITDVVNIGIGGSDLGPYMVTEALRPYKNHLNMHFVSNVDGTHIAEVLKKVNPETTLFLVASKTFTTQETMTNAHSARDWFLKAAGDEKHVAKHFAALSTNAKAVGEFGIDTANMFEFWDWVGGRYSLWSAIGLSIVLSIGFDNFVELLSGAHAMDKHFSTTPAEKNLPVLLALIGIWYNNFFGAETEAILPYDQYMHRFAAYFQQGNMESNGKYVDRNGNVVDYQTGPIIWGEPGTNGQHAFYQLIHQGTKMVPCDFIAPAITHNPLSDHHQKLLSNFFAQTEALAFGKSREVVEQEYRDQGKDPATLDYVVPFKVFEGNRPTNSILLREITPFSLGALIALYEHKIFTQGVILNIFTFDQWGVELGKQLANRILPELKDDKEISSHDSSTNGLINRYKAWRG

SEQ ID NO: 19 - DNA, 1476 bp, Escherichia coli
atggcggtaacgcaaacagcccaggcctgtgacctggtcattttcggcgcgaaaggcgaccttgcgcgtcgtaaattgctgccttccctgtatcaactggaaaaagccggtcagctcaacccggacacccggattatcggcgtagggcgtgctgactgggataaagcggcatataccaaagttgtccgcgaggcgctcgaaactttcatgaaagaaaccattgatgaaggtttatgggacaccctgagtgcacgtctggatttttgtaatctcgatgtcaatgacactgctgcattcagccgtctcggcgcgatgctggatcaaaaaaatcgtatcaccattaactactttgccatgccgcccagcacttttggcgcaatttgcaaagggcttggcgaggcaaaactgaatgctaaaccggcacgcgtagtcatggagaaaccgctggggacgtcgctggcgacctcgcaggaaatcaatgatcaggttggcgaatacttcgaggagtgccaggtttaccgtatcgaccactatcttggtaaagaaacggtgctgaacctgttggcgctgcgttttgctaactccctgtttgtgaataactgggacaatcgcaccattgatcatgttgagattaccgtggcagaagaagtggggatcgaagggcgctggggctattttgataaagccggtcagatgcgcgacatgatccagaaccacctgctgcaaattctttgcatgattgcgatgtctccgccgtctgacctgagcgcagacagcatccgcgatgaaaaagtgaaagtactgaagtctctgcgccgcatcgaccgctccaacgtacgcgaaaaaaccgtacgcgggcaatatactgcgggcttcgcccagggcaaaaaagtgccgggatatctggaagaagagggcgcgaacaagagcagcaatacagaaactttcgtggcgatccgcgtcgacattgataactggcgctgggccggtgtgccattctacctgcgtactggtaaacgtctgccgaccaaatgttctgaagtcgtggtctatttcaaaacacctgaactgaatctgtttaaagaatcgtggcaggatctgccgcagaataaactgactatccgtctgcaacctgatgaaggcgtggatatccaggtactgaataaagttcctggccttgaccacaaacataacctgcaaatcaccaagctggatctgagctattcagaaacctttaatcagacgcatctggcggatgcctatgaacgtttgctgctggaaaccatgcgtggtattcaggcactgtttgtacgtcgcgacgaagtggaagaagcctggaaatgggtagactccattactgaggcgtgggcgatggacaatgatgcgccgaaaccgtatcaggccggaacctggggacccgttgcctcggtggcgatgattacccgtgatggtcgttcctggaatgagtttgagtaa

SEQ ID NO: 20 - DNA, 1476 bp, Artificial Sequence
  Synthetic sequence, Glucose-6-phosphate dehydrogenase zwf (optimized)
atggcagttacccagaccgcacaggcatgtgatctggttatttttggtgcaaaaggtgatctggcacgtcgtaaactgctgccgagcctgtatcagctggaaaaagcaggtcagctgaatccggatacacgtattattggtgttggtcgtgcagattgggataaagcagcatataccaaagttgttcgtgaagcactggaaacctttatgaaagaaaccattgatgaaggtctgtgggataccctgagcgcacgtctggatttttgtaatctggatgttaatgataccgcagcatttagccgtctgggtgcaatgctggatcagaaaaatcgtattaccatcaactattttgcaatgcctccgagcacctttggtgccatttgtaaaggtctgggtgaagcaaaactgaatgcaaaaccggcacgtgttgttatggaaaaaccgctgggcaccagcctggcaaccagccaagaaattaatgatcaggtgggcgaatattttgaagagtgtcaggtttatcgcatcgatcattatctgggtaaagaaaccgttctgaatctgctggcactgcgttttgcaaatagcctgtttgtgaataactgggataatcgcaccattgatcatgtggaaattaccgttgcagaagaagttggtattgaaggtcgttggggctattttgataaagccggtcagatgcgtgatatgatccagaatcatctgctgcagattctgtgtatgattgcaatgagccctccgagcgatctgagcgcagatagcattcgtgatgaaaaagttaaagtgctgaaaagcctgcgtcgtattgatcgtagcaatgtgcgtgaaaaaaccgttcgtggtcagtataccgcaggttttgcacagggtaaaaaagttccgggttatctggaagaagaaggcgcaaataaaagcagtaataccgaaacctttgtggccattcgtgtggatattgataattggcgttgggcaggcgttccgttttatctgcgtaccggtaaacgtctgccgaccaaatgtagcgaagttgttgtttatttcaaaacaccggaactgaacctgtttaaagaaagctggcaggatctgccgcagaataaactgaccattcgtctgcagccggatgaaggtgttgatattcaggttctgaataaagttcctggcctggatcacaaacataacctgcagattaccaaactggatctgagctatagcgaaacgtttaatcagacccatctggcagatgcatatgaacgtctgctgctggaaaccatgcgtggtattcaggcactgtttgttcgccgtgatgaagttgaagaggcatggaaatgggttgatagcattaccgaagcatgggcaatggataatgatgcaccgaaaccgtatcaggcaggcacctggggtcctgttgcaagcgttgcaatgattacccgtgatggtcgtagctggaatgaatttgaataa

SEQ ID NO: 21 - protein, 491 aa, Escherichia coli
MAVTQTAQACDLVIFGAKGDLARRKLLPSLYQLEKAGQLNPDTRIIGVGRADWDKAAYTKVVREALETFMKETIDEGLWDTLSARLDFCNLDVNDTAAFSRLGAMLDQKNRITINYFAMPPSTFGAICKGLGEAKLNAKPARVVMEKPLGTSLATSQEINDQVGEYFEECQVYRIDHYLGKETVLNLLALRFANSLFVNNWDNRTIDHVEITVAEEVGIEGRWGYFDKAGQMRDMIQNHLLQILCMIAMSPPSDLSADSIRDEKVKVLKSLRRIDRSNVREKTVRGQYTAGFAQGKKVPGYLEEEGANKSSNTETFVAIRVDIDNWRWAGVPFYLRTGKRLPTKCSEVVVYFKTPELNLFKESWQDLPQNKLTIRLQPDEGVDIQVLNKVPGLDHKHNLQITKLDLSYSETFNQTHLADAYERLLLETMRGIQALFVRRDEVEEAWKWVDSITEAWAMDNDAPKPYQAGTWGPVASVAMITRDGRSWNEFE

SEQ ID NO: 22 - DNA, 996 bp, Escherichia coli
atgaagcaaacagtttatatcgccagccctgagagccagcaaattcacgtctggaatctgaatcatgaaggcgcactgacgctgacacaggttgtcgatgtgccggggcaggtgcagccgatggtggtcagcccggacaaacgttatctctatgttggtgttcgccctgagtttcgcgtcctggcgtatcgtatcgccccggacgatggcgcactgacctttgccgcagagtctgcgctgccgggtagtccgacgcatatttccaccgatcaccaggggcagtttgtctttgtaggttcttacaatgcgggtaacgtgagcgtaacgcgtctggaagatggcctgccagtgggcgtcgtcgatgtggtcgaggggctggacggttgccattccgccaatatctcaccggacaaccgtacgctgtgggttccggcattaaagcaggatcgcatttgcctgtttacggtcagcgatgatggtcatctcgtggcgcaggaccctgcggaagtgaccaccgttgaaggggccggcccgcgtcatatggtattccatccaaacgaacaatatgcgtattgcgtcaatgagttaaacagctcagtggatgtctgggaactgaaagatccgcacggtaatatcgaatgtgtccagacgctggatatgatgccggaaaacttctccgacacccgttgggcggctgatattcatatcaccccggatggtcgccatttatacgcctgcgaccgtaccgccagcctgattaccgttttcagcgtttcggaagatggcagcgtgttgagtaaagaaggcttccagccaacggaaacccagccgcgcggcttcaatgttgatcacagcggcaagtatctgattgccgccgggcaaaaatctcaccacatctcggtatacgaaattgttggcgagcaggggctactgcatgaaaaaggccgctatgcggtcgggcagggaccaatgtgggtggtggttaacgcacactaa

SEQ ID NO: 23 - DNA, 996 bp, Artificial Sequence
  Synthetic sequence, 6-Phosphoglucono lactonase pgl (optimized)
atgaaacagaccgtgtatattgcaagtccggaaagccagcagattcatgtttggaatctgaatcatgaaggtgcactgaccctgacacaggttgttgatgttccaggtcaggttcagccgatggttgttagtccggataaacgttatctgtatgttggtgttcgtccggaatttcgtgttctggcatatcgtattgcaccggatgatggtgccctgacctttgcagcagaaagcgcactgcctggtagcccgacacatatttcaaccgatcatcagggccagtttgtttttgttggtagctataatgcaggtaatgttagcgttacccgtctggaagatggtctgccggttggtgttgtggatgttgttgaaggtctggatggttgtcatagcgcaaatatttcaccggataatcgtaccctgtgggttcctgcactgaaacaggatcgtatttgtctgtttaccgttagtgatgatggtcatctggttgcacaggatccggcagaagttaccaccgttgaaggtgccggtccgcgtcatatggtttttcatccgaatgaacagtatgcctattgcgtgaatgaactgaatagcagcgttgatgtttgggaactgaaagatccgcatggtaatattgaatgtgttcagaccctggatatgatgccggaaaactttagtgatacccgttgggcagcagatattcatattactccggatggtcgtcatctgtatgcatgtgatcgtaccgcaagcctgattaccgtttttagcgttagcgaagatggtagcgttctgagcaaagaaggttttcagccgaccgaaacacagcctcgtggttttaatgttgatcacagcggtaaatatctgattgcagcaggtcagaaaagccatcatatttccgtttatgaaattgtgggtgaacagggtctgctgcatgaaaaaggtcgttatgcagttggtcagggtccgatgtgggttgttgttaatgcacattaa

SEQ ID NO: 24 - protein, 331 aa, Escherichia coli
MKQTVYIASPESQQIHVWNLNHEGALTLTQVVDVPGQVQPMVVSPDKRYLYVGVRPEFRVLAYRIAPDDGALTFAAESALPGSPTHISTDHQGQFVFVGSYNAGNVSVTRLEDGLPVGVVDVVEGLDGCHSANISPDNRTLWVPALKQDRICLFTVSDDGHLVAQDPAEVTTVEGAGPRHMVFHPNEQYAYCVNELNSSVDVWELKDPHGNIECVQTLDMMPENFSDTRWAADIHITPDGRHLYACDRTASLITVFSVSEDGSVLSKEGFQPTETQPRGFNVDHSGKYLIAAGQKSHHISVYEIVGEQGLLHEKGRYAVGQGPMWVVVNAH

SEQ ID NO: 25 - DNA, 1407 bp, Escherichia coli
atgtccaagcaacagatcggcgtagtcggtatggcagtgatgggacgcaaccttgcgctcaacatcgaaagccgtggttataccgtctctattttcaaccgttcccgtgagaagacggaagaagtgattgccgaaaatccaggcaagaaactggttccttactatacggtgaaagagtttgtcgaatctctggaaacgcctcgtcgcatcctgttaatggtgaaagcaggtgcaggcacggatgctgctattgattccctcaaaccatatctcgataaaggagacatcatcattgatggtggtaacaccttcttccaggacactattcgtcgtaatcgtgagctttcagcagagggctttaacttcatcggtaccggtgtttctggcggtgaagagggggcgctgaaaggtccttctattatgcctggtggccagaaagaagcctatgaattggtagcaccgatcctgaccaaaatcgccgccgtagctgaagacggtgaaccatgcgttacctatattggtgccgatggcgcaggtcactatgtgaagatggttcacaacggtattgaatacggcgatatgcagctgattgctgaagcctattctctgcttaaaggtggcctgaacctcaccaacgaagaactggcgcagacctttaccgagtggaataacggtgaactgagcagttacctgatcgacatcaccaaagatatcttcaccaaaaaagatgaagacggtaactacctggttgatgtgatcctggatgaagcggctaacaaaggtaccggtaaatggaccagccagagcgcgctggatctcggcgaaccgctgtcgctgattaccgagtctgtgtttgcacgttatatctcttctctgaaagatcagcgtgttgccgcatctaaagttctctctggtccgcaagcacagccagcaggcgacaaggctgagttcatcgaaaaagttcgtcgtgcgctgtatctgggcaaaatcgtttcttacgcccagggcttctctcagctgcgtgctgcgtctgaagagtacaactgggatctgaactacggcgaaatcgcgaagattttccgtgctggctgcatcatccgtgcgcagttcctgcagaaaatcaccgatgcttatgccgaaaatccacagatcgctaacctgttgctggctccgtacttcaagcaaattgccgatgactaccagcaggcgctgcgtgatgtcgttgcttatgcagtacagaacggtattccggttccgaccttctccgcagcggttgcctattacgacagctaccgtgctgctgttctgcctgcgaacctgatccaggcacagcgtgactattttggtgcgcatacttataagcgtattgataaagaaggtgtgttccataccgaatggctggattaa

SEQ ID NO: 26 - DNA, 1407 bp, Artificial Sequence
  Synthetic sequence, 6-Phosphogluconate dehydrogenase gnd (optimized)
atgagcaaacagcagattggtgttgttggtatggcagttatgggtcgtaatctggcactgaatattgaaagccgtggttataccgtgagcatttttaaccgtagccgtgaaaaaaccgaagaagtgattgcagaaaatccgggtaaaaaactggttccgtattacaccgttaaagagtttgttgaaagcctggaaacaccgcgtcgtattctgctgatggttaaagccggtgcaggcaccgatgcagcaattgatagcctgaaaccgtatctggataaaggcgatattatcattgatggtggcaacacctttttccaggataccattcgtcgtaatcgtgaactgagcgcagaaggctttaactttattggcaccggtgttagcggtggtgaagaaggtgcactgaaaggtccgagcattatgcctggtggtcagaaagaagcatacgaactggttgcaccgattctgaccaaaattgcagcagttgccgaagatggtgaaccgtgtgttacctatattggtgcagatggtgcaggtcattatgtgaaaatggtgcataacggtatcgagtatggtgatatgcagctgattgcggaagcatatagcctgctgaaaggtggtctgaatctgaccaatgaagaactggcacagacctttaccgaatggaataatggtgaactgtccagctatctgatcgatatcaccaaagacatcttcaccaaaaaagatgaggatggcaattatctggtggatgtgattctggatgaagcagcaaataaaggcaccggtaaatggaccagccagagcgcactggatctgggtgaaccgctgagcctgattaccgaaagcgtttttgcacgttatatcagcagcctgaaagatcagcgtgttgcagcaagcaaagttctgagcggtccgcaggcacagcctgccggtgataaagcagaatttattgaaaaagttcgccgtgcgctgtatctgggtaaaattgttagctatgcacagggttttagccagctgcgtgcagccagcgaagaatacaattgggatctgaattatggcgagatcgccaaaatctttcgtgccggttgtattattcgtgcacagtttttacagaaaatcaccgatgcctatgccgaaaatccgcagattgcaaacctgctgctggcaccgtattttaagcagattgccgatgattatcagcaggcactgcgtgatgttgttgcatatgccgttcagaatggtattccggttccgacctttagcgcagccgttgcatattatgatagttatcgtgcagccgttctgcctgcaaatctgattcaggcccagcgtgattattttggtgcacatacctataaacgcatcgataaagaaggtgtgtttcatacagaatggctggactaa

SEQ ID NO: 27 - protein, 468 aa, Escherichia coli
MSKQQIGVVGMAVMGRNLALNIESRGYTVSIFNRSREKTEEVIAENPGKKLVPYYTVKEFVESLETPRRILLMVKAGAGTDAAIDSLKPYLDKGDIIIDGGNTFFQDTIRRNRELSAEGFNFIGTGVSGGEEGALKGPSIMPGGQKEAYELVAPILTKIAAVAEDGEPCVTYIGADGAGHYVKMVHNGIEYGDMQLIAEAYSLLKGGLNLTNEELAQTFTEWNNGELSSYLIDITKDIFTKKDEDGNYLVDVILDEAANKGTGKWTSQSALDLGEPLSLITESVFARYISSLKDQRVAASKVLSGPQAQPAGDKAEFIEKVRRALYLGKIVSYAQGFSQLRAASEEYNWDLNYGEIAKIFRAGCIIRAQFLQKITDAYAENPQIANLLLAPYFKQIADDYQQALRDVVAYAVQNGIPVPTFSAAVAYYDSYRAAVLPANLIQAQRDYFGAHTYKRIDKEGVFHTEWLD

SEQ ID NO: 28 - DNA, 660 bp, Escherichia coli
atgacgcaggatgaattgaaaaaagcagtaggatgggcggcacttcagtatgttcagcccggcaccattgttggtgtaggtacaggttccaccgccgcacactttattgacgcgctcggtacaatgaaaggccagattgaaggggccgtttccagttcagatgcttccactgaaaaactgaaaagcctcggcattcacgtttttgatctcaacgaagtcgacagccttggcatctacgttgatggcgcagatgaaatcaacggccacatgcaaatgatcaaaggcggcggcgcggcgctgacccgtgaaaaaatcattgcttcggttgcagaaaaatttatctgtattgcagacgcttccaagcaggttgatattctgggtaaattcccgctgccagtagaagttatcccgatggcacgtagtgcagtggcgcgtcagctggtgaaactgggcggtcgtccggaataccgtcagggcgtggtgaccgataatggcaacgtgatcctcgacgtccacggcatggaaatccttgacccgatagcgatggaaaacgccataaatgcgattcctggcgtggtgactgttggcttgtttgctaaccgtggcgcggacgttgcgctgattggcacacctgacggtgtcaaaaccattgtgaaatga

SEQ ID NO: 29 - DNA, 660 bp, Artificial Sequence
  Synthetic sequence, Ribose-5-phosphate isomerase rpiA (optimized)
atgacccaggatgaactgaaaaaagcagttggttgggcagcactgcagtatgttcagcctggcaccattgttggtgttggcaccggtagcaccgcagcacattttattgatgcactgggcaccatgaaaggtcagattgaaggtgcagttagcagcagtgatgcaagcaccgaaaaactgaaaagcctgggtattcatgtgtttgatctgaatgaagttgatagcctgggcatttatgttgatggtgccgatgaaattaatggccatatgcagatgattaaaggtggtggtgcagcactgacccgtgaaaaaatcattgcaagcgttgccgaaaagtttatctgtattgcagatgccagcaaacaggttgatattctgggtaaatttccgctgccggttgaagttattccgatggcacgtagcgcagttgcacgtcagctggttaaacttggtggtcgtccggaatatcgtcagggtgttgttaccgataatggtaatgttattctggatgtgcatggcatggaaattctggatccgattgcaatggaaaatgccattaatgcaattccgggtgttgtgacagttggtctgtttgcaaatcgtggtgcagatgttgcactgattggtacaccggatggtgttaaaaccattgtgaaataa

SEQ ID NO: 30 - protein, 219 aa, Escherichia coli
MTQDELKKAVGWAALQYVQPGTIVGVGTGSTAAHFIDALGTMKGQIEGAVSSSDASTEKLKSLGIHVFDLNEVDSLGIYVDGADEINGHMQMIKGGGAALTREKIIASVAEKFICIADASKQVDILGKFPLPVEVIPMARSAVARQLVKLGGRPEYRQGVVTDNGNVILDVHGMEILDPIAMENAINAIPGVVTVGLFANRGADVALIGTPDGVKTIVK

SEQ ID NO: 31 - DNA, 450 bp, Escherichia coli
atgaaaaagattgcatttggctgtgatcatgtcggtttcattttaaaacatgaaatagtggcacatttagttgagcgtggcgttgaagtgattgataaaggaacctggtcgtcagagcgtactgattatccacattacgccagtcaagtcgcactggctgttgctggcggagaggttgatggcgggattttgatttgtggtactggcgtcggtatttcgatagcggcgaacaagtttgccggaattcgcgcggtcgtctgtagcgaaccttattccgcgcaactttcgcggcagcataacgacaccaacgtgctggcttttggttcacgagtggttggcctcgaactggcaaaaatgattgtggatgcgtggctgggcgcacagtacgaaggcggtcgtcatcaacaacgcgtggaggcgattacggcaatagagcagcggagaaattga

SEQ ID NO: 32 - DNA, 450 bp, Artificial Sequence
  Synthetic sequence, Ribose-5-phosphate isomerase rpiB (optimized)
atgaaaaaaatcgcctttggctgcgatcatgtgggctttattctgaaacatgaaattgttgcccatctggttgaacgtggtgttgaagttattgataaaggcacctggtcaagcgaacgtaccgattatccgcattatgcaagccaggttgcactggcagttgccggtggtgaagttgatggtggtattctgatttgtggcaccggtgttggtattagcattgcagcaaacaaatttgcaggtattcgtgcagttgtttgtagcgaaccgtatagcgcacagctgagccgtcagcataatgataccaatgttctggcatttggtagccgtgttgttggtctggaactggcaaaaatgattgttgatgcatggctgggtgcacagtatgaaggtggtcgtcatcagcagcgtgttgaagcaattaccgcaattgaacagcgtcgcaattaa

SEQ ID NO: 33 - protein, 149 aa, Escherichia coli
MKKIAFGCDHVGFILKHEIVAHLVERGVEVIDKGTWSSERTDYPHYASQVALAVAGGEVDGGILICGTGVGISIAANKFAGIRAVVCSEPYSAQLSRQHNDTNVLAFGSRVVGLELAKMIVDAWLGAQYEGGRHQQRVEAITAIEQRRN

SEQ ID NO: 34 - DNA, 948 bp, Escherichia coli
gtgcctgatatgaagctttttgctggtaacgccaccccggaactagcacaacgtattgccaaccgcctgtacacttcactcggcgacgccgctgtaggtcgctttagcgatggcgaagtcagcgtacaaattaatgaaaatgtacgcggtggtgatattttcatcatccagtccacttgtgcccctactaacgacaacctgatggaattagtcgttatggttgatgccctgcgtcgtgcttccgcaggtcgtatcaccgctgttatcccctactttggctatgcgcgccaggaccgtcgcgtccgttccgctcgtgtaccaatcactgcgaaagtggttgcagacttcctctccagcgtcggtgttgaccgtgtgctgacagtggatctgcacgctgaacagattcagggtttcttcgacgttccggttgataacgtatttggtagcccgatcctgctggaagacatgctgcagctgaatctggataacccaattgtggtttctccggacatcggcggcgttgtgcgtgcccgcgctatcgctaagctgctgaacgataccgatatggcaatcatcgacaaacgtcgtccgcgtgcgaacgtttcacaggtgatgcatatcatcggtgacgttgcaggtcgtgactgcgtactggtcgatgatatgatcgacactggcggtacgctgtgtaaagctgctgaagctctgaaagaacgtggtgctaaacgtgtatttgcgtacgcgactcacccgatcttctctggcaacgcggcgaacaacctgcgtaactctgtaattgatgaagtcgttgtctgcgataccattccgctgagcgatgaaatcaaatcactgccgaacgtgcgtactctgaccctgtcaggtatgctggccgaagcgattcgtcgtatcagcaacgaagaatcgatctctgccatgttcgaacactaa

SEQ ID NO: 35 - DNA, 948 bp, Artificial Sequence
  Synthetic sequence, Phosphoribosyl pyrophosphate synthetase prs (optimized)
gtgccggatatgaaactgtttgcaggtaatgcaacaccggaactggcacagcgtattgcaaatcgtctgtataccagcctgggtgatgcagcagttggtcgttttagtgatggtgaagttagcgtgcagattaatgaaaatgttcgcggtggcgatatctttattatccagagcacctgtgcaccgaccaatgataatctgatggaactggttgttatggttgatgcactgcgtcgtgcaagcgcaggtcgtattaccgcagttattccgtattttggttatgcacgtcaggatcgtcgtgttcgtagcgcacgtgttccgattaccgcaaaagttgttgcagattttctgagcagcgttggtgttgatcgtgttctgaccgttgatctgcatgccgagcagattcagggtttttttgatgttccggtggataatgtttttggtagcccgattctgctggaagatatgctgcagctgaatctggataacccgattgttgttagtccggatattggtggtgttgtgcgtgcacgtgccattgcaaaactgctgaatgataccgatatggcgattattgataaacgtcgtccgcgtgcaaatgttagccaggttatgcatataattggtgatgttgcaggtcgtgattgtgttctggtggatgatatgattgataccggtggcaccctgtgtaaagcagccgaagcactgaaagaacgtggtgcaaaacgtgtttttgcctatgcaacccatccgatttttagcggtaatgcagcaaataatctgcgcaatagcgttattgatgaagttgttgtttgtgacaccattccgctgagtgatgaaatcaaaagcctgccgaatgttcgtaccctgacactgagcggtatgctggcagaagcaattcgtcgtattagcaatgaagaaagcattagcgccatgtttgaacattaa

SEQ ID NO: 36 - protein, 315 aa, Escherichia coli
MPDMKLFAGNATPELAQRIANRLYTSLGDAAVGRFSDGEVSVQINENVRGGDIFIIQSTCAPTNDNLMELVVMVDALRRASAGRITAVIPYFGYARQDRRVRSARVPITAKVVADFLSSVGVDRVLTVDLHAEQIQGFFDVPVDNVFGSPILLEDMLQLNLDNPIVVSPDIGGVVRARAIAKLLNDTDMAIIDKRRPRANVSQVMHIIGDVAGRDCVLVDDMIDTGGTLCKAAEALKERGAKRVFAYATHPIFSGNAANNLRNSVIDEVVVCDTIPLSDEIKSLPNVRTLTLSGMLAEAIRRISNEESISAMFEH

SEQ ID NO: 37 - DNA, 1476 bp, Homo sapiens
atgaatcctgcggcagaagccgagttcaacatcctcctggccaccgactcctacaaggttactcactataaacaatatccacccaacacaagcaaagtttattcctactttgaatgccgtgaaaagaagacagaaaactccaaattaaggaaggtgaaatatgaggaaacagtattttatgggttgcagtacattcttaataagtacttaaaaggtaaagtagtaaccaaagagaaaatccaggaagccaaagatgtctacaaagaacatttccaagatgatgtctttaatgaaaagggatggaactacattcttgagaagtatgatgggcatcttccaatagaaataaaagctgttcctgagggctttgtcattcccagaggaaatgttctcttcacggtggaaaacacagatccagagtgttactggcttacaaattggattgagactattcttgttcagtcctggtatccaatcacagtggccacaaattctagagagcagaagaaaatattggccaaatatttgttagaaacttctggtaacttagatggtctggaatacaagttacatgattttggctacagaggagtctcttcccaagagactgctggcataggagcatctgctcacttggttaacttcaaaggaacagatacagtagcaggacttgctctaattaaaaaatattatggaacgaaagatcctgttccaggctattctgttccagcagcagaacacagtaccataacagcttgggggaaagaccatgaaaaagatgcttttgaacatattgtaacacagttttcatcagtgcctgtatctgtggtcagcgatagctatgacatttataatgcgtgtgagaaaatatggggtgaagatctaagacatttaatagtatcaagaagtacacaggcaccactaataatcagacctgattctggaaaccctcttgacactgtgttaaaggttttggagattttaggtaagaagtttcctgttactgagaactcaaagggttacaagttgctgccaccttatcttagagttattcaaggggatggagtagatattaataccttacaagagattgtagaaggcatgaaacaaaaaatgtggagtattgaaaatattgccttcggttctggtggaggtttgctacagaagttgacaagagatctcttgaattgttccttcaagtgtagctatgttgtaactaatggccttgggattaacgtcttcaaggacccagttgctgatcccaacaaaaggtccaaaaagggccgattatctttacataggacgccagcagggaattttgttacactggaggaaggaaaaggagaccttgaggaatatggtcaggatcttctccatactgtcttcaagaatggcaaggtgacaaaaagctattcatttgatgaaataagaaaaaatgcacagctgaatattgaactggaagcagcacatcattag

SEQ ID NO: 38 - DNA, 1476 bp, Artificial Sequence
  Synthetic sequence, Nicotinamide phosphoribosyltransferase HS (optimized)
atgaatccggcagcagaagccgaatttaacattctgctggcaaccgatagctataaagtgacccattataaacagtatccgcctaataccagcaaagtgtatagctattttgagtgccgtgagaaaaaaaccgaaaacagcaaactgcgcaaagtgaaatatgaagaaaccgtgttttatggcctgcagtacatcctgaacaaatacctgaaaggtaaagtggtgaccaaagagaaaattcaagaggccaaagatgtgtataaagaacactttcaggatgacgtgttcaacgaaaaaggctggaactatatcctggaaaaatatgatggtcatctgccgattgaaattaaagcagttccggaaggttttgttattccgcgtggtaatgttctgtttaccgttgaaaataccgatccggaatgttattggctgaccaattggattgaaaccattctggttcagagctggtatccgattaccgttgcaaccaatagccgtgaacagaaaaaaatcctggccaaatatctgctggaaaccagcggtaatctggatggtctggaatacaaactgcatgattttggttatcgtggtgttagcagccaagaaaccgcaggtattggtgcaagcgcacatctggttaactttaaaggcaccgataccgtggcaggtctggcactgattaaaaagtattatggcaccaaagatccggttccgggttatagcgttccggcagccgaacattcaaccattaccgcatggggtaaagatcatgaaaaagatgcctttgaacatatcgtgacccagtttagcagcgtgccggttagcgttgttagcgatagttatgatatctataatgcctgcgagaagatctggggtgaagatctgcgtcatctgattgttagccgtagcacccaggcaccgctgattattcgtccggatagtggtaatccgctggataccgttctgaaagttctggaaattctgggcaaaaaattcccggttacggaaaatagcaaaggctataaactgctgcctccgtatctgcgtgttattcaaggtgatggtgtggatattaacaccctgcaagaaattgtggaaggcatgaaacagaaaatgtggtccattgaaaatatcgcctttggtagcggtggtggtctgctgcagaaactgacccgtgatctgctgaattgtagctttaaatgcagctatgttgtgaccaatggtctgggcattaacgtttttaaagatcctgttgccgatccgaataaacgcagcaaaaaaggtcgtctgagcctgcatcgtacaccggcaggtaattttgttaccctggaagaaggtaaaggcgatctggaagaatatggtcaggatctgctgcataccgttttcaaaaatggcaaagtgaccaaaagctacagctttgatgaaattcgtaaaaacgcccagctgaacattgaactggaagcagcacatcattaa

SEQ ID NO: 39 - protein, 491 aa, Homo sapiens [sequence omitted]

SEQ ID NO: 40 - DNA, 48 bp, Artificial Sequence
  Synthetic sequence, Primer for NAMPT CP (forward)
aggagatataccatgaccaaagaaaacctgattctgctggcagatgca

SEQ ID NO: 41 - DNA, 48 bp, Artificial Sequence
  Synthetic sequence, Primer for NAMPT CP (reverse)
gctcgaattcggatcttagatggttgcgtttttacggatctgctcaaa

SEQ ID NO: 42 - DNA, 48 bp, Artificial Sequence
  Synthetic sequence, Primer for NAMPT SSC (forward)
aggagatataccatgaagaatctgattctggccaccgatagctataaa

SEQ ID NO: 43 - DNA, 48 bp, Artificial Sequence
  Synthetic sequence, Primer for NAMPT SSC (reverse)
gctcgaattcggatcttaacgaccttcgctacgtttacgaactgcatc

SEQ ID NO: 44 - DNA, 48 bp, Artificial Sequence
  Synthetic sequence, Primer for NAMPT HS (forward)
aggagatataccatgaatccggcagcagaagccgaatttaacattctg

SEQ ID NO: 45 - DNA, 48 bp, Artificial Sequence
  Synthetic sequence, Primer for NAMPT HS (reverse)
gctcgaattcggatcttaatgatgtgctgcttccagttcaatgttcag

SEQ ID NO: 46 - DNA, 70 bp, Artificial Sequence
  Synthetic sequence, Primer for pgi to pCDF (forward)
cgtgatggtcgtagctggaatgaatttgaataaaaggagatataccatgaagaacattaatccgacacag

SEQ ID NO: 47 - DNA, 70 bp, Artificial Sequence
  Synthetic sequence, Primer for pgi to pCDF (reverse)
acttaagcattatgcggccgcaagcttgtcgacctgcaggcgcgccgttaaccacgccaggctttataac

SEQ ID NO: 48 - DNA, 70 bp, Artificial Sequence
  Synthetic sequence, Primer for zwf to pCDF (forward)
gtcagggtccgatgtgggttgttgttaatgcacattaaaaggagatataccatggcagttacccagaccg

SEQ ID NO: 49 - DNA, 24 bp, Artificial Sequence
  Synthetic sequence, Primer for zwf to pCDF (reverse)
ttattcaaattcattccagctacg

SEQ ID NO: 50 - DNA, 70 bp, Artificial Sequence
  Synthetic sequence, Primer for pgl to pCDF (forward)
agaaggtgtgtttcatacagaatggctggactaaaaggagatataccatgaaacagaccgtgtatattgc

SEQ ID NO: 51 - DNA, 23 bp, Artificial Sequence
  Synthetic sequence, Primer for pgl to pCDF (reverse)
ttaatgtgcattaacaacaaccc

SEQ ID NO: 52 - DNA, 70 bp, Artificial Sequence
  Synthetic sequence, Primer for gnd to pCDF (forward)
tggtacaccggatggtgttaaaaccattgtgaaataaaaggagatataccatgagcaaacagcagattgg

SEQ ID NO: 53 - DNA, 70 bp, Artificial Sequence
  Synthetic sequence, Primer for gnd to pCDF (reverse)
cattatgcggccgcaagcttgtcgacctgcaggcgcgccgagctcttagtccagccattctgtatgaaac

SEQ ID NO: 54 - DNA, 70 bp, Artificial Sequence
  Synthetic sequence, Primer for rpiA to pCDF (forward)
caattaccgcaattgaacagcgtcgcaattaaaaggagatataccatgacccaggatgaactgaaaaaag

SEQ ID NO: 55 - DNA, 27 bp, Artificial Sequence
  Synthetic sequence, Primer for rpiA to pCDF (reverse)
ttatttcacaatggttttaacaccatc

SEQ ID NO: 56 - DNA, 70 bp, Artificial Sequence
  Synthetic sequence, Primer for rpiB to pCDF (forward)
aatgaagaaagcattagcgccatgtttgaacattaaaaggagatataccatgaaaaaaatcgcctttggc

SEQ ID NO: 57 - DNA, 18 bp, Artificial Sequence
  Synthetic sequence, Primer for rpiB to pCDF (reverse)
ttaattgcgacgctgttc

SEQ ID NO: 58 - DNA, 70 bp, Artificial Sequence
  Synthetic sequence, Primer for prs to pCDF (forward)
attcccctgtagaaataattttgtttaactttaataaggagatataccgtgccggatatgaaactgtttg

SEQ ID NO: 59 - DNA, 20 bp, Artificial Sequence
  Synthetic sequence, Primer for prs to pCDF (reverse)
ttaatgttcaaacatggcgc

SEQ ID NO: 60 - DNA, 70 bp, Artificial Sequence
  Synthetic sequence, Primer for pgi to pACYC (forward)
tcccctgtagaaataattttgtttaactttaataaggagatataccatgaagaacattaatccgacacag

SEQ ID NO: 61 - DNA, 23 bp, Artificial Sequence
  Synthetic sequence, Primer for pgi to pACYC (reverse)
ttaaccacgccaggctttataac

SEQ ID NO: 62 - DNA, 70 bp, Artificial Sequence
  Synthetic sequence, Primer for zwf to pACYC (forward)
atggtctgattaatcgttataaagcctggcgtggttaaaaggagatataccatggcagttacccagaccg

SEQ ID NO: 63 - DNA, 24 bp, Artificial Sequence
  Synthetic sequence, Primer for zwf to pACYC (reverse)
ttattcaaattcattccagctacg

SEQ ID NO: 64 - DNA, 70 bp, Artificial Sequence
  Synthetic sequence, Primer for pgl to pACYC (forward)
ccgtgatggtcgtagctggaatgaatttgaataaaaggagatataccatgaaacagaccgtgtatattgc

SEQ ID NO: 65 - DNA, 23 bp, Artificial Sequence
  Synthetic sequence, Primer for pgl to pACYC (reverse)
ttaatgtgcattaacaacaaccc

SEQ ID NO: 66 - DNA, 70 bp, Artificial Sequence
  Synthetic sequence, Primer for gnd to pACYC (forward)
tcagggtccgatgtgggttgttgttaatgcacattaaaaggagatataccatgagcaaacagcagattgg

SEQ ID NO: 67 - DNA, 70 bp, Artificial Sequence; identical to SEQ ID NO: 53
  Synthetic sequence, Primer for gnd to pACYC (reverse)

SEQ ID NO: 68 - DNA, 70 bp, Artificial Sequence
  Synthetic sequence, Primer for rpiA to pACYC (forward)
aaggtgtgtttcatacagaatggctggactaaaaggagatataccatgacccaggatgaactgaaaaaag

SEQ ID NO: 69 - DNA, 27 bp, Artificial Sequence
  Synthetic sequence, Primer for rpiA to pACYC (reverse)
ttatttcacaatggttttaacaccatc

SEQ ID NO: 70 - DNA, 70 bp, Artificial Sequence
  Synthetic sequence, Primer for rpiB to pACYC (forward)
ggtacaccggatggtgttaaaaccattgtgaaataaaaggagatataccatgaaaaaaatcgcctttggc

SEQ ID NO: 71 - DNA, 18 bp, Artificial Sequence
  Synthetic sequence, Primer for rpiB to pACYC (reverse)
ttaattgcgacgctgttc

SEQ ID NO: 72 - DNA, 70 bp, Artificial Sequence
  Synthetic sequence, Primer for prs to pACYC (forward)
aagcaattaccgcaattgaacagcgtcgcaattaaaaggagatataccgtgccggatatgaaactgtttg

SEQ ID NO: 73 - DNA, 70 bp, Artificial Sequence
  Synthetic sequence, Primer for prs to pACYC (reverse)
tcgacttaagcattatgcggccgcaagcttgtcgacctgcaggcgcgccgttaatgttcaaacatggcgc

SEQ ID NO: 74 - DNA, 32 bp, Artificial Sequence
  Synthetic sequence, Primer for niaP (forward)
aggagatataccatgcctgcagcaaccgcacc

SEQ ID NO: 75 - DNA, 50 bp, Artificial Sequence
  Synthetic sequence, Primer for niaP (reverse)
gctcgaattcggatcttagcttgctttatctgctgctgttgccggataac

SEQ ID NO: 76 - DNA, 49 bp, Artificial Sequence
  Synthetic sequence, Primer for niaX (forward)
aggagatataccttgagcggtctgctgtatcacaccagcgtttatgcag

SEQ ID NO: 77 - DNA, 49 bp, Artificial Sequence
  Synthetic sequence, Primer for niaX (reverse)
gctcgaattcggatcttagcgacgtttacgcagaactttataaactgcc

SEQ ID NO: 78 - DNA, 48 bp, Artificial Sequence
  Synthetic sequence, Primer for pnuC (forward)
aggagatataccatggttcgtagtccgctgtttctgctgattagcagc

SEQ ID NO: 79 - DNA, 51 bp, Artificial Sequence
  Synthetic sequence, Primer for pnuC (reverse)
gctcgaattcggatcttagatgtagttgttcacgcgttcacgttctttatg

SEQ ID NO: 80 - DNA, 66 bp, Artificial Sequence
  Synthetic sequence, Primer part2 for pnuC BM (forward)
tattagttaagtataagaaggagatatacaatggttcgtagtccgctgtttctgctgattagcagc

SEQ ID NO: 81 - DNA, 66 bp, Artificial Sequence
  Synthetic sequence, Primer part2 for pnuC BM (reverse)
atgctagttattgctcagcggtggcagcagttagatgtagttgttcacgcgttcacgttctttatg

SEQ ID NO: 82 - DNA, 50 bp, Artificial Sequence
  Synthetic sequence, Primer part2 for niaP BC (forward)
tattagttaagtataagaaggagatatacaatgcctgcagcaaccgcacc

SEQ ID NO: 83 - DNA, 65 bp, Artificial Sequence
  Synthetic sequence, Primer part2 for niaP BC (reverse)
atgctagttattgctcagcggtggcagcagttagcttgctttatctgctgctgttgccggataac

(19) United States
(12) Patent Application Publication
(10) Pub. No.: US 2022/0056458 A1
(43) Pub. Date: Feb. 24, 2022

(54) GENETICALLY MODIFIED MICROORGANISM AND METHOD BOTH FOR PRODUCING NICOTINAMIDE DERIVATIVE, AND VECTOR FOR USE IN SAME

(71) Applicants: TEIJIN LIMITED, Osaka-shi, Osaka (JP); SYNART CO., LTD., Kobe-shi, Hyogo (JP)

(72) Inventors: Shinichiro SHOJI, Kobe-shi (JP); Jun ISHII, Kobe-shi (JP); Akihiko KONDO, Kobe-shi (JP); Hidekazu WATANABE, Osaka-shi (JP); Masanobu KANOU, Osaka-shi (JP); Ryota NAKAJIMA, Osaka-shi (JP)

(73) Assignees: TEIJIN LIMITED, Osaka-shi, Osaka (JP); SYNART CO., LTD., Kobe-shi, Hyogo (JP)

(21) Appl. No.: 17/415,328

(22) PCT Filed: Dec. 17, 2019
(86) PCT No.: PCT/JP2019/049479

§ 371 Date: Jun. 17, 2021

(30) Foreign Application Priority Data
Dec. 18, 2018 (JP) 2018-236634

(51) Int. Cl. C12N 15/70; C12P 19/36; C12P 19/32; C12N 9/04; C12N 9/92; C12N 9/18; C12N 9/10

(52) U.S. Cl. CPC C12N 15/70; C12P 19/36; C12P 19/32; C12N 9/0006; C12N 9/92; C12N 9/18; C12Y 204/02045; C12Y 503/01009; C12Y 101/01049; C12Y 301/01031; C12Y 101/01043; C12Y 503/01006; C12N 9/1077

(57) ABSTRACT

Provided is a technique for synthesizing a nicotinamide derivative (NAm derivative) such as a nicotinamide mononucleotide (NMN) with high efficiency. A genetically modified microorganism is used, which can express, as nicotinamide phosphoribosylt ransferase (NAMPT), NAMPT having a conversion efficiency of 5-folds or more that of human NAMPT.

### TECHNICAL FIELD

The present invention relates to a novel recombinant microorganism for producing a nicotinamide derivative (NAm derivative) such as nicotinamide mononucleotide (NMN) and a novel method for producing a NAm derivative, as well as novel vectors for use in the same.

### BACKGROUND ART

Nicotinamide adenine dinucleotide (NAD) is a nucleotide derived from ribose and nicotinamide. NAD functions as a coenzyme in various redox reactions in vivo and is known to play a central role in aerobic respiration (oxidative phosphorylation). NAD can take two forms in vivo, an oxidized form (NAD⁺) and a reduced form (NADH). The term “NAD” herein encompasses both the oxidized (NAD⁺) and reduced (NADH) forms, unless otherwise specified.

There are multiple biosynthetic pathways for NAD, of which the main pathway in mammalian cells is the one that uses nicotinamide (NAm) as a starting material from which NAD is synthesized through a two-step enzymatic reaction. In the first step, NAm taken up into the cell is converted by nicotinamide phosphoribosyl transferase (NAMPT: NMN synthase) in the presence of 5-phosphoribosyl-1-pyrophosphate (PRPP) into nicotinamide mononucleotide (NMN) and pyrophosphate (P-Pi). In the subsequent second step, NMN obtained in the previous step is converted to NAD by nicotinamide/nicotinate mononucleotide adenylyltransferase (NMNAT) in the presence of adenosine triphosphate (ATP).

NMN, which plays a role as a precursor of NAD in the above biosynthetic pathway, is known to have various functions such as activating mitochondria and sirtuin genes, which are so-called longevity genes. It is considered that especially in vivo, the decrease in NMN production capacity with aging leads to a decrease in NAD, a decrease in mitochondrial activity, and damage to the cell nucleus. NMN has also been reported to involve in aging-related diseases, such as insulin resistance, diabetes, cancer, and Alzheimer's disease. Based on these findings, NMN is attracting attention as a research tool, an intermediate in the synthesis of NAD, and an active pharmaceutical ingredient.

NMN is expected to be used as a synthetic intermediate not only for NAD but also for various nicotinamide derivatives (NAm derivatives), such as nicotinamide riboside (NR) and nicotinate mononucleotide (NaMN).

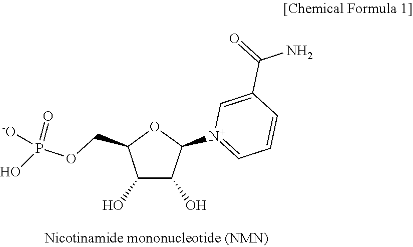

Conventional methods for the synthesis of NMN include the organic synthesis method, the method based on degradation of NAD, and the synthetic biological method using microorganisms.

According to the organic synthesis method, NMN is synthesized from D-ribose through several steps (Patent Literature 1: US2018/0291054A). This method is time-consuming and costly because it requires several steps of synthesis.

According to the NAD degradation method, NAD biosynthesized by yeast is enzymatically degraded without isolation (Patent Literature 2: WO2017/200050A). This method has a drawback of very poor productivity of NMN per bacterial cell.

According to the synthetic biological method using microorganisms, a host microorganism such as E. coli is genetically engineered to thereby construct a recombinant microorganism that expresses an enzyme similar to biological enzymes that catalyze the first step in the main biosynthetic system of NAD in mammals, i.e., the step of converting NAm and PRPP to NMN (NAMPT: NMN synthase), and the resulting recombinant microorganism is used for synthesis of NMN (Patent Literature 3: WO2015/069860A; Non-Patent Literature 1: Mariescu et al., Scientific Reports, Aug. 16, 2018, Vol. 8, No. 1, p. 12278). This method cannot achieve sufficient productivity for practical use, since it requires a long time for NMN synthesis but can yield only a small amount of NMN.

### LIST OF CITATIONS

### Patent Literature

[Patent Literature 1] US2018/0291054A
[Patent Literature 2] WO2017/200050A
[Patent Literature 3] WO2015/069860A

### Non-Patent Literature

[Non-Patent Literature 1] Mariescu et al., Scientific reports, Aug. 16, 2018, Vol. 8, No. 1, pp. 12278

### SUMMARY OF INVENTION

### Problem to be Solved by the Invention

With the above backgrounds, there is a need for efficient synthesis methods of nicotinamide derivatives (NAm derivatives) such as nicotinamide mononucleotide (NMN).

### Means to Solve the Problem

In light of the above issues, the present inventors examined the conventional synthetic biological production system of NMN using microorganisms and, as a result of intensive investigation to improve it, have found that the production efficiency of NMN can be significantly improved by genetically engineering the host microorganism to express a specific enzyme with enhanced activity as a key enzyme (NAMPT) for the biosynthesis of NMN, whereby the production efficiency of NMN can be improved. The present inventors have also found that the production efficiency of NMN can be improved further by genetically engineering the host microorganism to express one or more specific enzymes with enhanced activity as a protein that promotes the uptake of a reactant of NMN synthesis, NAm, or a derivative thereof, NA, into the host microorganism cell (niacin transporter) and/or as a protein that promotes the excretion of NMN or other NAm derivatives out of the host microorganism cell (NAm derivative transporter), whereby the uptake efficiency of NAm into the host microorganism cell can be improved. The present inventors have further found that the production efficiency of NMN can be improved still further by genetically engineering the host microorganism to express one or more specific enzymes with enhanced activity as one or more of a series of enzymes constituting the biosynthetic system of PRPP, another reactant of NMN synthesis, whereby the synthesis efficiency of PRPP can be improved. The present inventors have also found that the thus-improved NMN system can be utilized for producing not only NMN but also other NAm derivatives such as NAD, NR, and NaMN with high efficiency. The present invention has been completed based on these new findings.

The present invention may involve the following aspects.

[1] A recombinant microorganism for producing a nicotinamide derivative, wherein the microorganism has been engineered to express a nicotinamide phosphoribosyl transferase (NAMPT), which converts nicotinamide and phosphoribosyl pyrophosphate into nicotinamide mononucleotide, and/or has been transformed with a vector carrying a nucleic acid encoding the amino acid sequence of the NAMPT, wherein the conversion efficiency of the NAMPT from nicotinamide to nicotinamide mononucleotide is five times or more of the conversion efficiency of a human NAMPT.
[2] The recombinant microorganism according to [1], wherein the NAMPT is composed of a polypeptide with a sequence homology of 80% or more with the amino acid sequence represented by SEQ ID NO:3 or SEQ ID NO:6.
[3] The recombinant microorganism according to [1] or [2], wherein the microorganism has been engineered to express a niacin transporter, which promotes cellular uptake of nicotinic acid and/or nicotinamide, and/or has been transformed with a vector carrying a nucleic acid encoding the amino acid sequence of the niacin transporter, wherein the niacin transporter increases the intracellular uptake efficiency of nicotinic acid and/or nicotinamide by the host microorganism by 1.1 times or more.
[4] The recombinant microorganism according to [3], wherein the niacin transporter is composed of a polypeptide with a sequence homology of 80% or more with the amino acid sequence represented by SEQ ID NO:9 or SEQ ID NO:12.
[5] The recombinant microorganism according to any one of [1] to [4], wherein the microorganism has been engineered to express a nicotinamide derivative transporter, which promotes extracellular excretion of a nicotinamide derivative and/or has been transformed with a vector carrying a nucleic acid encoding the amino acid sequence of the nicotinamide derivative transporter, wherein the nicotinamide derivative transporter increases extracellular excretion efficiency of the nicotinamide derivative by the host microorganism by 3 times or more.
[6] The recombinant microorganism according to [5], wherein the nicotinamide derivative transporter is composed of a polypeptide with a sequence homology of 80% or more with the amino acid sequence represented by SEQ ID NO:15.
[7] The recombinant microorganism according to any one of [1] to [6], wherein the microorganism has been engineered to express one or more enzymes which promote a synthetic pathway from glucose-6-phosphoric acid to phosphoribosyl pyrophosphate and/or has been transformed with a vector carrying a nucleic acid encoding the amino acid sequence of the one or more enzymes.
[8] The recombinant microorganism according to [7], wherein the one or more enzymes are selected from the group consisting of phosphoglucose isomerase, glucose-6-phosphate dehydrogenase, 6-phosphogluconolactonase, 6-phosphogluconate dehydrogenase, ribose-5-phosphate isomerase, and phosphoribosyl pyrophosphate synthase.
[9] The recombinant microorganism according to [8], wherein

the phosphoglucose isomerase is a polypeptide with a sequence homology of 80% or more with the amino acid sequence represented by SEQ ID NO:18,

the glucose-6-phosphate dehydrogenase is a polypeptide with a sequence homology of 80% or more with the amino acid sequence represented by SEQ ID NO:21,

the 6-phosphogluconolactonase is a polypeptide with a sequence homology of 80% or more with the amino acid sequence represented by SEQ ID NO:24,

the 6-phosphogluconate dehydrogenase is a polypeptide with a sequence homology of 80% or more with the amino acid sequence represented by SEQ ID NO:27,

the ribose-5-phosphate isomerase is a polypeptide with a sequence homology of 80% or more with the amino acid sequence represented by SEQ ID NO:30 or SEQ ID NO33, and

the phosphoribosyl pyrophosphate synthase is a polypeptide with a sequence homology of 80% or more with the amino acid sequence represented by SEQ ID NO:36.

[10] The recombinant microorganism according to any one of [1] to [9], wherein the nicotinamide derivative is selected from the group consisting of nicotinamide mononucleotide, nicotinamide adenine dinucleotide, nicotinamide riboside, nicotinate mononucleotide, nicotinamide adenine dinucleotide phosphoric acid, and nicotinate adenine dinucleotide.
[11] The recombinant microorganism according to any one of [1] to [10], wherein the recombinant microorganism is E. coli or a yeast.
[12] A method for producing a nicotinamide derivative, comprising:

providing nicotinamide to a recombinant microorganism according to any one of Aspects 1 to 11; and

recovering the nicotinamide derivative produced by the microorganism.

[13] The method according to [12], further comprising purifying the recovered nicotinamide derivative.
[14] A vector carrying a nucleic acid encoding the amino acid sequence of nicotinamide phosphoribosyl transferase (NAMPT), which converts nicotinamide and phosphoribosyl pyrophosphate into nicotinamide mononucleotide, wherein the nucleic acid comprises a base sequence with a sequence identity of 80% or more with the base sequence represented by SEQ ID NO:2 or SEQ ID NO:5.
[15] A vector carrying a nucleic acid encoding the amino acid sequence of a niacin transporter, which promotes cellular uptake of nicotinic acid and/or nicotinamide, wherein the nucleic acid comprises a base sequence with a sequence identity of 80% or more with the base sequence represented by SEQ ID NO:8 or SEQ ID NO:11.
[16] A vector carrying a nucleic acid encoding the amino acid sequence of a nicotinamide derivative transporter, which promotes extracellular excretion of a nicotinamide derivative, wherein the nucleic acid comprises a base sequence with a sequence identity of 80% or more with the base sequence represented by SEQ ID NO:14.

### Advantageous Effects of Invention

The present invention allows for efficient synthesis of NAm derivatives such as NMN.

### BRIEF DESCRIPTION OF DRAWINGS

FIG. 1 is a schematic diagram showing an example of a synthetic biological production system for NAm derivatives.

### DESCRIPTION OF EMBODIMENTS

The present invention will be described in detail in accordance with specific embodiments indicated below. However, the present invention should not be restricted to any of the embodiments disclosed below, but can be implemented in any form to the extent that it does not deviate from the gist of the present invention.

All patent publications, patent application publications, and non-patent documents cited herein are hereby incorporated into this disclosure by reference in their entirety for all purposes.

The term “nucleic acid” used herein encompasses ribonucleic acids, deoxyribonucleic acids, or any modifications of such nucleic acids, and also encompasses both single-stranded and double-stranded nucleic acids. Any nucleic acids (genes) disclosed herein can be prepared by any method known to those skilled in the art, using primers or probes prepared either consulting databases of public organizations known to those skilled in the art or using sequences disclosed herein. Such nucleic acids (genes) can be easily obtained as cDNA of the genes, for example, by using various types of PCR and other DNA amplification techniques known to those skilled in the art. Alternatively, a person skilled in the art can synthesize a nucleic acid using any conventional technique as appropriate based on the sequence information disclosed herein.

The statement that any nucleic acid or gene “encodes” a protein or polypeptide herein means that the nucleic acid or gene expresses the protein or polypeptide with maintaining its activities. The term “encode” herein means encoding a protein disclosed herein either as a continuous structural sequence (exon) or in the form of two or more discrete segments with one or more appropriate intervening sequences (introns).

Gene engineering techniques mentioned herein, such as cloning of nucleic acids or genes, design and preparation of vectors, transformation of cells, and expression of proteins or polypeptides, can be carried out with reference to, e.g., Sambrook, J. et al, Molecular Cloning: A Laboratory Manual, 2nd Ed. Manual, 2nd Ed., Cold Spring Harbor Laboratory Press, Cold Spring Harbor, N.Y., 1989.

### I. Overview

This chapter provides an overview of the present invention.

As mentioned above, in an attempt to produce nicotinamide mononucleotide (NMN) via synthetic biological using microorganisms, a method has been proposed which involves constructing a recombinant microorganism expressing enzymes similar to those constituting the main biosynthetic system of nicotinamide adenine dinucleotide (NAD) in mammals by genetically engineering a host microorganism such as E. coli, and using the resulting recombinant microorganism to synthesize NMN (Patent Literature 3: WO2015/069860A; Non-Patent Literature 1: Mariescu et al., Scientific reports, Aug. 16, 2018, Vol. 8, No. 1, pp. 12278). However, this conventional method has a drawback in that it cannot achieve sufficient productivity for practical use, since it requires a long time for NMN synthesis but can yield only a small amount of NMN.

The present invention relates to a NAm derivative synthesis system derived from the conventional synthetic biological production system of NMN using microorganisms, with improved production efficiency of NAm derivatives such as NMN, by genetically engineering a microorganism to express various enzymes and/or transporter proteins involved in the synthesis and/or transport of NAm derivatives.

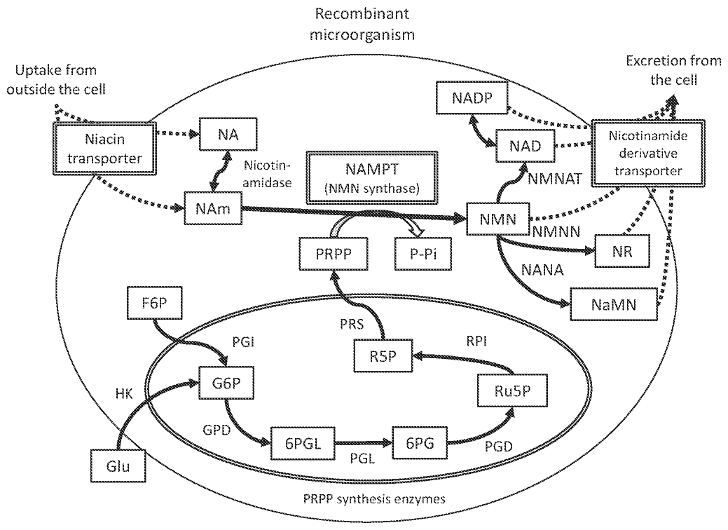

An exemplary synthetic biological production system for NAm derivatives according to an embodiment of the present invention will be described in more detail using FIG. 1. Note that FIG. 1 merely illustrates an example, while the present invention should not be limited to the synthesis system shown in FIG. 1.

The synthetic biological system of NAm-derivatives shown in FIG. 1 includes a NAm-derivative synthesis system consisting of a series of enzymes in a host microorganism cell. As shown in FIG. 1, the main reaction pathway of the NMN synthesis system is the reaction pathway that converts NAm and PRPP into NMN and P-Pi.

NAm, one of the two reactants of the main reaction pathway of the NAm derivative synthesis system, is taken into the host microorganism cell from outside, and is interconvertible by nicotinamidase with NAm, which is also taken into the host microorganism cell from outside.

PRPP, the other reactant of the main reaction pathway of the NAm derivative synthesis system, is synthesized from glucose (Glu) taken into the host microorganism cell from outside, through the following series of reaction steps.

Phosphorylation of glucose (Glu) to glucose-6-phosphate (G6P) by hexokinase (HK).

Conversion of fructose-6-phosphate (F6P) to glucose-6-phosphate (G6P) by phosphoglucose isomerase (PGI).

Conversion of G6P to 6-phosphoglucono-1,5-lactone (6PGL) by glucose-6-phosphate dehydrogenase (GPD).

Conversion of 6PGL to 6-phosphogluconate (6PG) by 6-phosphogluconolactonase (PGL).

Conversion of 6PG to ribulose-5-phosphate (Ru5P) by 6-phosphogluconate dehydrogenase (PGD).

Conversion of Ru5P to ribose-5-phosphate (R5P) by ribose-5-phosphate isomerase (RPI).

Conversion of R5P to 5-phosphoribosyl-1-pyrophosphate (PRPP) by phosphoribosyl pyrophosphate synthase (PRS).

The NMN produced by the main reaction mentioned above is then converted to NAD by NMNAT, to nicotinate mononucleotide (NaMN) by nicotinamide nucleotide amidase (NANA), and to nicotinamide riboside by nicotinamide mononucleotide-5-nucleotidase (NMNN), which are then excreted from the cell as appropriate.

According to an aspect of the present invention, a host microorganism is genetically engineered to express a specific enzyme with improved activity as a key enzyme for the biosynthesis of NMN (NAMPT: NMN synthase) to enhance the efficiency of NMN synthesis, thereby improving the production efficiency of NAm derivatives.

According to a preferred aspect of the present invention, the NAm derivative synthesis system mentioned above is modified by genetically engineering the host microorganism to express a specific protein with improved activity as a transporter protein (niacin transporter) that promotes the intracellular uptake of NAm and/or NA (niacin) to enhance the efficiency of niacin uptake into the host microorganism cell, thereby further improving the production efficiency of NAm derivatives.

According to another preferred aspect of the present invention, the NAm derivative synthesis system mentioned above is modified by genetically engineering the host microorganism to express a specific enzyme with improved activity as one or more of the series of enzymes (GPI, GPD, PGL, PGD, RPI, and PRS) that constitute the biosynthetic system of PRPP, another reactant of NMN synthesis, to enhance the efficiency of PRPP synthesis, thereby further improving the production efficiency of NAm derivatives.

According to another preferred aspect of the present invention, the NAm derivative synthesis system mentioned above is modified by genetically engineering the host microorganism to express a specific protein with improved activity as a transporter protein (NAm derivative transporter) that promotes the extracellular excretion of NAm derivatives to enhance the efficiency of excretion of the produced NAm derivatives from host microorganism cell, thereby further improving the production efficiency of NAm derivatives.

According to the present invention, the overall production efficiency of the NAm derivative can be significantly improved by introducing a combination of two or more, preferably all, of the specific NAMPT (NMN synthase), niacin transporter, NAm derivative transporter, and PRPP synthesis enzymes (GPI, GPD, PGL, PGD, RPI, and PRS) into the NAm derivative synthesis system of the recombinant microorganism.

NAm derivatives that can be produced by the synthetic system of the present invention aside from NMN include, but are not limited to, NAD, nicotinamide riboside (NR), nicotinate mononucleotide (NaMN), nicotinamide adenine dinucleotide phosphate (NADP), nicotinate adenine dinucleotide, etc.

While the next and subsequent chapters will be given mainly to an embodiment of the synthetic system of the present invention for synthesizing NMN, other embodiments relating to the synthesis of NAm derivatives other than NMN are briefly described below.

For example, synthesis of NAD by the synthetic system of the present invention can be achieved by genetically engineering the host microorganism to express, in addition to the series of genes for NMN synthesis, nicotinamide/nicotinic acid mononucleotide adenylyltransferase (NMNAT), which converts NMN to NAD, according to the same procedure as described above to enhance the conversion efficiency of NMN to NAD.

Synthesis of NADP by the synthetic system of the present invention can be achieved by genetically engineering the host microorganism to express, in addition to the series of genes for NMN synthesis, the NMNAT mentioned above along with a NAD+ kinase, which converts NAD to NADP, according to the same procedure as described above to enhance the conversion efficiency of NMN to NAD and NAD to NADP.

Synthesis of NR by the synthetic system of the present invention can be achieved by genetically engineering the host microorganism to express, in addition to the series of genes for NMN synthesis, nicotinamide mononucleotide-5-nucleotidase (NMNN), which further converts NMN to NR, according to the same procedure as described above to enhance the conversion efficiency of NMN to NR.

Synthesis of NaMN by the synthetic system can also be achieved by genetically engineering the host microorganism to express, in addition to the series of genes for NMN synthesis, nicotinamide nucleotide amidase (NANA), which further converts NMN to NaMN, according to the same procedure as described above to enhance the conversion efficiency of NMN to NaMN.

According to the method using the NAm derivative synthesis system of the present invention with the constitution described above, the production efficiency of NAm derivatives such as NMN can be significantly improved, compared to the conventional synthetic biological production method of NMN. For example, the results shown in the Examples below show that the method using the NAm derivative synthesis system of the present invention can produce more than 10 times the amount of NMN compared to the amount of NMN produced by the conventional synthetic biological methods. The method using the NAm derivative synthesis system of the present invention is also advantageous in that it involves reduced amounts of by-products and has excellent selectivity for NMN and other NAm derivatives.

### II. Enzymes

This chapter deals with the enzymes involved in the production of NAm derivatives used in the present invention.

The term “homology” between two amino acid sequences herein means the ratio of identical or similar amino acid residues appearing in each corresponding position when these amino acid sequences are aligned, and the term “identity” between two amino acid sequences herein means the ratio of identical amino acid residues appearing in each corresponding position when these amino acid sequences are aligned.

The “homology” and “identity” between two amino acid sequences can be determined, e.g., with the Needle program of the EMBOSS package (EMBOSS: The European Molecular Biology Open Software Suite, Rice et al., 2000, Trends Genet. 16: 276-277) (preferably version 5.00 or later), using the Needleman-Wunsch algorithm (Needleman and Wunsch, 1970, J. Mol. Biol. 48: 443-453).

Similar amino acids herein include, e.g., amino acids that belong to the same group in the classification based on structures, characteristics, and types of side chains indicated below.

Aromatic amino acids: F, H, W, Y;

Aliphatic amino acids: I, L, V;

Hydrophobic amino acids: A, C, F, H, I, K, L, M, T, V, W, Y;

Charged amino acids: D, E, H, K, R, etc.;

Positively charged amino acids: H, K, R;

Negatively charged amino acids: D, E;

Polar amino acids: C, D, E, H, K, N, Q, R, S, T, W, Y;

Small amino acids: A, C, D, G, N, P, S, T, V, etc.;

Very small amino acids: A, C, G. S;

Amino acids with aliphatic side chains: G, A, V, L, I:

Amino acids with aromatic side chains: F, Y, W;

Amino acids with sulphur-containing side chains: C, M;

Amino acids with aliphatic hydroxyl side chains: S, T;

Amino acids with basic side chains: K, R, H; and

Acidic amino acids and their amide derivatives: D, E, N, Q.

(1) Enzyme that Catalyzes the Synthesis of NMN from NAm and PRPP (NMN Synthase):

Various enzymes derived from various microorganisms have been known as NMN synthases that catalyze the synthesis of NMN from NAm and PRPP (NAMPTs). Such known enzymes can be selectively used as appropriate.

According to the present invention, a specific enzyme with improved activity may preferably be used as NMN synthase (NAMPT). One reason is that an NAMPT with poor activity may decrease the selectivity of NMN production from the substrates, i.e., NAm and PRPP. Another reason is that an NAMPT with poor activity may require a longer time for producing NMN and may lead to a decrease in the production rate due to the degradation and conversion of NMN. Such an NMN synthase with improved activity may be referred to herein as “the NMN synthase of the present invention,” although NMN synthases (NAMPT) that can be used in the present invention are not limited to the one explained below.

Specifically, the NMN synthase (NAMPT) of the present invention may preferably have a conversion efficiency of NAm to NMN (NAMPT conversion efficiency) that is typically 5-fold or higher, particularly 7-fold or higher, more particularly 9-fold or higher, compared to the NAMPT conversion efficiency of human NAMPT. As shown in the [EXAMPLES] section below, according to the inventors' investigation, no production of NMN was observed in a bacterium expressing NAMPT with 2-fold or 3-fold conversion efficiency as well as in a bacterium expressing human NAMPT, while a significant increase in NMN production was observed in a bacterium expressing NAMPT with 6-fold conversion efficiency compared to the bacterium expressing human NAMPT.

A specific example of a method for measuring the NAMPT conversion efficiency of NAMPT is as follows: Escherichia coli (hereinafter referred as E. coli) BL21 (DE3) strain is transformed with a plasmid derived from pRSFDuet-1 via incorporation of a gene for expressing NAMPT to prepare a construct strain, which is then inoculated into a test tube containing 5 mL of LB medium and cultured at 37° C. at 200 rpm for 12 hours. The culture is then inoculated into a 500 ml conical flask containing 200 ml of LB medium such that the resulting OD₆₀₀ is 0.03, and incubated at 37° C. at 200 rpm. When OD₆₀₀ reached 0.4, isopropyl-β-thiogalactopyranoside is added such that the final concentration becomes 0.1 mM, and the culture is further incubated at 25° C. at 200 rpm for 16 hours. 30 mL of the culture medium is then transferred to a 50 mL conical tube, centrifuged at 3000 g for 5 minutes, and the bacterial cells are collected. The cells are washed with 1×PBS, the wash fluid is centrifuged at 3000 g for 5 minutes, and the bacterial cells are collected. This operation is repeated twice. The recovered bacterial cells are suspended in 15 mL of Cell Lysis Buffer (MBL) to prepare a bacterial lysate solution (lysate) according to a generally recommended method. The bacterial lysate is measured for OD₅₉₅ using Protein Assay Bradford Reagent (Wako Pure Chemical Co., Ltd.), and then diluted with water such that the OD₅₉₅ value becomes 0.1. The resulting diluted solution is used as NAMPT solution and subjected to the measurement of NAMPT activity according to the One-Step Assay Method of CycLexR NAMPT Colorimetric Assay Kit Ver. 2 (MBL). The measurement can be carried out using SpectraMaxR iD3 multimode microplate reader (Molecular Devices Inc.) or other instruments. According to the present invention, the absorbance at 450 nm is measured every 5 minutes at 30° C. for up to 60 minutes, the absorbance values at three points where the slope is at its maximum value are selected, and their slope is defined as the NAMPT conversion efficiency.

However, the measurement method of NAMPT conversion efficiency is not limited to the specific example explained above, but may be any other evaluation method so long as it gives equivalent values.

Among them, the NMN synthase of the present invention may preferably be an enzyme comprising a polypeptide having the amino acid sequence represented by SEQ ID NO:3 or SEQ ID NO:6, or a polypeptide having a similar amino acid sequence thereto.

The amino acid sequence of an NAMPT from Sphingopyxis sp. C-I strain is shown in SEQ ID NO:3, and the nucleotide sequence of the naturally-occurring gene encoding the NAMPT of SEQ ID NO:3 is shown in SEQ ID NO:1. The present inventors optimized the nucleotide sequence of the naturally-occurring gene of SEQ ID NO:1 so as to improve its expression and activity in the host microorganism. The resulting optimized nucleotide sequence encoding the NAMPT of SEQ ID NO:3 is shown in SEQ ID NO:2.

The amino acid sequence of an NAMPT from Chitinophaga pinensis is shown in SEQ ID NO:6, and the nucleotide sequence of the naturally-occurring gene encoding the NAMPT of SEQ ID NO:6 is shown in SEQ ID NO:4. The present inventors optimized the nucleotide sequence of the naturally-occurring gene of SEQ ID NO:4 so as to improve its expression and activity in the host microorganism. The resulting optimized nucleotide sequence encoding the NAMPT of SEQ ID NO:6 is shown in SEQ ID NO:5.

Specifically, the NMN synthase of the present invention may preferably have a polypeptide with an amino acid sequence with a homology (preferably identity) of 80% or more, particularly 85% or more, still particularly 90% or more, even still particularly 95% or more, or 96% or more, or 97% or more, or 99% or more, especially 100%, to the amino acid sequence shown in SEQ ID NO:3 or SEQ ID NO:6.

(2) Transporter Protein that Promotes the Intracellular Uptake of Niacin (Niacin Transporter):

Various transporter proteins derived from various microorganisms have been known as transporters that promote the intracellular uptake of NAm and/or NA (niacin) (niacin transporters). Such known transporter proteins can be selectively used as appropriate.

According to the present invention, a specific transporter protein with improved activity may preferably be used as a niacin transporter. One reason is that a niacin transporter with poor niacin uptake efficiency may lead to a low intracellular abundance of NAm that reacts with PRPP, thus resulting in a poor selectivity of NMN production from the substrates NAm and PRPP. Another reason is that a niacin transporter with poor niacin uptake efficiency may require a longer time for producing NMN and may lead to a decrease in the production rate due to the degradation and conversion of NMN. Such a transporter protein with improved niacin uptake efficiency may be referred to herein as “the niacin transporter of the present invention,” although niacin transporters that can be used in the present invention are not limited to the one explained below.

Specifically, the niacin transporter of the present invention may preferably increase the efficiency of intracellular uptake of nicotinic acid and/or nicotinamide (niacin uptake efficiency) by the host microorganism typically by 1.1-fold or more, particularly by 1.2-fold or more, compared to the niacin uptake efficiency of the host microorganism that does not express the niacin transporter of the present invention. Use of such a niacin transporter with an improved niacin uptake efficiency results in a higher intracellular abundance of NAm in response to its concentration compared to the host microorganism that does not express the niacin transporter of the present invention, resulting in an increase in NMN production.

A specific example of a method for measuring the niacin uptake efficiency of a niacin transporter is as follows: Escherichia coli (E. Coli) BL21 (DE3) strain is genetically engineered to express NAMPT with a NAMPT conversion efficiency of 200 or higher, and the resulting strain is then transformed with a plasmid derived from pCDFDuet-1 by inserting E. coli (E. Coli) K12-derived genes prs, rpiB, rpiA, gnd, pgl, zwf, and pgi so as to express these genes in this order. The resultant transformant strain is further transformed with another plasmid derived pACYCDuet-1 via incorporation of a gene encoding the niacin transporter to prepare a construct strain, which is then inoculated into a test tube containing 5 mL of LB medium and cultured at 37° C. at 200 rpm for 12 hours. The culture is then inoculated into a 500 ml conical flask containing 200 ml of LB medium such that the resulting OD₆₀₀ is 0.03, and incubated at 37° C. at 200 rpm. When OD₆₀₀ reached 0.4, isopropyl-β-thiogalactopyranoside is added such that the final concentration becomes 0.1 mM, and the culture is further incubated at 25° C. at 200 rpm for 16 hours. The culture medium is then transferred to a 50 mL conical tube, centrifuged at 3000 g for 5 minutes, and the bacterial cells are collected. The cells are washed with 1×PBS, the wash fluid is centrifuged at 3000 g for 5 minutes, and the bacterial cells are collected. This operation is repeated twice. The collected bacterial cells are suspended in LB medium to obtain an OD₆₀₀ of 10, 10 mL of which suspension is transferred to a 100-mL conical flask, and combined with nicotinamide at 1 g/L, D-glucose at 0.4 g/L, and phosphate buffer (pH 6.2) at 0.005 mol/L, and the reaction is allowed to run at 30° C. at 200 rpm. The reaction solution is collected 1 hour and 2 hours from the start of the reaction. The collected solutions are frozen at −30° C., thawed, and centrifuged at 12,000 rpm for 3 minutes to collect the supernatant. These liquid samples are analyzed by HPLC to quantify the amounts of NMN. The obtained NMN quantitative values is used for determining the niacin uptake efficiency of niacin transporter according to Equation (1) below.

[Formula 1]

Niacin uptake efficiency of the niacin transporter (%)={(NMN amount after 1 hour of reaction)/(NMN amount after 2 hour of reaction)}×100 Equation (1)

However, the measurement method of the niacin uptake efficiency of a niacin transporter is not limited to the specific example explained above, but may be any other evaluation method so long as it gives equivalent values.

Among them, the niacin transporter of the present invention may preferably be a protein comprising a polypeptide having the amino acid sequence represented by SEQ ID NO:9 or SEQ ID NO:12, or a polypeptide having a similar amino acid sequence thereto.

The amino acid sequence of the niacin transporter from Burkholderia cenocepacia is shown in SEQ ID NO:9, and the nucleotide sequence of the naturally-occurring gene encoding the niacin transporter of SEQ ID NO:9 is shown in SEQ ID NO:7. The present inventors optimized the nucleotide sequence of the naturally-occurring gene of SEQ ID NO:7 so as to improve its expression and activity in the host microorganism. The resulting optimized nucleotide sequence encoding the niacin transporter of SEQ ID NO:9 is shown in SEQ ID NO:8.

The amino acid sequence of the niacin transporter from Streptococcus pneumoniae is shown in SEQ ID NO:12, and the nucleotide sequence of the naturally-occurring gene encoding the niacin transporter of SEQ ID NO:12 is shown in SEQ ID NO:10. The present inventors optimized the nucleotide sequence of the naturally-occurring gene of SEQ ID NO:10 so as to improve its expression and activity in the host microorganism. The resulting optimized nucleotide sequence encoding the niacin transporter of SEQ ID NO:12 is shown in SEQ ID NO:11.

Specifically, the niacin transporter of the present invention may preferably have a polypeptide with an amino acid sequence with a homology (preferably identity) of 80% or more, particularly 85% or more, still particularly 90% or more, even still particularly 95% or more, or 96% or more, or 97% or more, or 99% or more, especially 100%, to the amino acid sequence shown in SEQ ID NO:9 or SEQ ID NO:12.

(3) Transporter Protein that Promotes the Extracellular Export of NAm Derivatives (NAm Derivative Transporter):

Various transporters derived from various microorganisms have been known as transporters that promote the extracellular export of NMN, which is a product of NMN synthesis, and/or NAm derivatives such as NR and NaMN, which are synthesized from NMN (NAm derivative transporters). Such known transporter proteins can be selectively used as appropriate.

According to the present invention, a specific transporter protein with improved activity may preferably be used as an NAm derivative transporter. One reason is that an NAm derivative transporter with poor NAm derivative excretion efficiency may leave a substantial amount of NMN decomposed and/or converted in the cell, resulting in a decrease in NMN production. Another reason is that an NAm derivative transporter with improved excretion efficiency may serve to accelerate the extracellular excretion of the produced NAm derivatives and thereby facilitate the recovery process of the produced NAm derivatives. Such a transporter protein with improved NAm derivative excretion efficiency may be referred to herein as “the NAm derivative transporter of the present invention,” although NAm derivative transporters that can be used in the present invention are not limited to the one explained below.

Specifically, the NAm derivative transporter of the present invention may preferably increase the efficiency of extracellular excretion of nicotinamide derivatives (NAm derivative excretion efficiency) by the host microorganism by usually 3-fold or more, particularly 5-fold or more, even more particularly 7-fold or more, compared to the NAm derivative excretion efficiency of the host microorganism that does not express the NAm derivative transporter of the present invention.

A specific example of a method for measuring the NAm derivative excretion efficiency of a NAm derivative transporter, in the case of a transporter that transports NMN as an NAm derivative (i.e., NMN transporter), is as follows: Escherichia coli (E. coli) BL21 (DE3) strain is genetically engineered to express NAMPT with a NAMPT conversion efficiency of 200 or higher, and the resulting strain is then transformed with a plasmid derived from pCDFDuet-1 by inserting E. coli (E. Coli) K12-derived genes prs, rpiB, rpiA, gnd, pgl, zwf, and pgi so as to express these genes in this order. The resultant transformant strain is further transformed with another plasmid derived pACYCDuet-1 via incorporation of a gene encoding the NMN transporter to prepare a construct strain, which is then inoculated into a test tube containing 5 mL of LB medium and cultured at 37° C. at 200 rpm for 12 hours. The culture is then inoculated into a 500 ml conical flask containing 200 ml of LB medium such that the resulting OD₆₀₀ is 0.03, and incubated at 37° C. at 200 rpm. When OD₆₀₀ reached 0.4, isopropyl-β-thiogalactopyranoside is added such that the final concentration becomes 0.1 mM, and the culture is further incubated at 25° C. at 200 rpm for 16 hours. The culture medium is then transferred to a 50 mL conical tube, centrifuged at 3000 g for 5 minutes, and the bacterial cells are collected. The tube is washed with 1×PBS, the wash fluid is centrifuged at 3000 g for 5 minutes, and the bacterial cells are collected. This operation is repeated twice. The collected bacterial cells are suspended in LB medium to obtain an OD₆₀₀ of 10, 10 mL of which suspension is transferred to a 100-mL conical flask, and combined with nicotinamide at 1 g/L, r-glucose at 0.4 g/L, and phosphate buffer (pH 6.2) at 0.005 mol/L, and the reaction is allowed to run at 30° C. at 200 rpm. The reaction solution is collected 2 hours from the start of the reaction. The collected solution is divided into two, one of which is frozen at −30° C., thawed, and centrifuged at 12,000 rpm for 3 minutes to collect the supernatant, and the other is not frozen but is directly centrifuged at 12,000 rpm for 3 minutes to collect the supernatant. These liquid samples are analyzed by HPLC to quantify the amounts of NMN. The obtained NMN quantitative values is used for determining the NMN excretion efficiency of NMN transporter according to Equation (2) below.

[Formula 2]

NMN excretion efficiency of the NMN transporter (%)={(NMN amount without frozen treatment)/(NMN amount with frozen treatment)}×100 Equation (2)

In the case of NAm derivative transporters that transport NAm derivatives other than NMN, the excretion efficiency of NAm derivatives can be determined in accordance with a method similar to the one explained above.

However, the measurement method of the NAm derivative excretion efficiency of an NAm derivative transporter is not limited to the specific example explained above, but may be any other evaluation method so long as it gives equivalent values.

Among them, the NAm derivative transporter of the present invention may preferably be a protein comprising a polypeptide having the amino acid sequence represented by SEQ ID NO:13, or a polypeptide having a similar amino acid sequence thereto.

The amino acid sequence of the NAm derivative transporter (NMN transporter) from Bacillus mycoides is shown in SEQ ID NO:15, and the nucleotide sequence of the naturally-occurring gene encoding the NAm derivative transporter of SEQ ID NO:15 is shown in SEQ ID NO:13. The present inventors optimized the nucleotide sequence of the naturally-occurring gene of SEQ ID NO:13 so as to improve its expression and activity in the host microorganism. The resulting optimized nucleotide sequence encoding the NAm derivative transporter of SEQ ID NO:15 is shown in SEQ ID NO:14.

Specifically, the NAm derivative transporter of the present invention may preferably have a polypeptide with an amino acid sequence with a homology (preferably identity) of 80% or more, particularly 85% or more, still particularly 90% or more, even still particularly 95% or more, or 96% or more, or 97% or more, or 99% or more, especially 100%, to the amino acid sequence shown in SEQ ID NO:15.

(4) Enzymes Involved in the Synthesis of PRPP from G6P (PGI, GPD, PGL, PGD, RPI, and PRS):

As the enzymes involved in the synthesis of PRPP from G6P, namely phosphoglucose isomerase (PGI), glucose 6-phosphate dehydrogenase (GPD), 6-phosphogluconolactonase (PGL), 6-phosphogluconate dehydrogenase (PGD), ribose-5-phosphate isomerase (RPI), and phosphoribosyl pyrophosphate synthase (PRS) (collectively referred to as “PRPP synthesis-related enzymes” as appropriate), various enzymes derived from various microorganisms have been known, and have also been optimized according to various host microorganisms. Such known enzymes can be selectively used as appropriate.

Examples of PRPP synthesis-related enzymes particularly preferred for use in the present invention are listed below. However, PRPP synthesis-related enzymes that can be used in the present invention are not limited to these examples.

Phosphoglucose isomerase (PGI) may be an enzyme comprising a polypeptide having the amino acid sequence shown in SEQ ID NO:18, or a polypeptide having a similar amino acid sequence thereto.

The amino acid sequence of the enzyme pgi from E. coli is shown in SEQ ID NO:18, and the nucleotide sequence of the naturally-occurring gene encoding the enzyme pgi of SEQ ID NO:18 is shown in SEQ ID NO:16. The present inventors optimized the nucleotide sequence of the naturally-occurring gene of SEQ ID NO:16 so as to improve its expression and activity in the host microorganism. The resulting optimized nucleotide sequence encoding the enzyme pgi of SEQ ID NO:18 is shown in SEQ ID NO:17.

Specifically, the enzyme pgi may preferably have a polypeptide with an amino acid sequence with a homology (preferably identity) of 80% or more, particularly 85% or more, still particularly 90% or more, even still particularly 95% or more, or 96% or more, or 97% or more, or 99% or more, especially 100%, to the amino acid sequence shown in SEQ ID NO:18.

Glucose 6-phosphate dehydrogenase (GPD) may be an enzyme comprising a polypeptide having the amino acid sequence shown in SEQ ID NO:21, or a polypeptide having a similar amino acid sequence thereto.

The amino acid sequence of the enzyme zwf from E. coli is shown in SEQ ID NO:21, and the nucleotide sequence of the naturally-occurring gene encoding the enzyme zwf of SEQ ID NO:21 is shown in SEQ ID NO:19. The present inventors optimized the nucleotide sequence of the naturally-occurring gene of SEQ ID NO:19 so as to improve its expression and activity in the host microorganism. The resulting optimized nucleotide sequence encoding the enzyme zwf of SEQ ID NO:21 is shown in SEQ ID NO:20.

Specifically, the enzyme GPD may preferably have a polypeptide with an amino acid sequence with a homology (preferably identity) of 80% or more, particularly 85% or more, still particularly 90% or more, even still particularly 95% or more, or 96% or more, or 97% or more, or 99% or more, especially 100%, to the amino acid sequence shown in SEQ ID NO:21.

6-Phosphogluconolactonase (PGL) may be an enzyme comprising a polypeptide having the amino acid sequence shown in SEQ ID NO:24, or a polypeptide having a similar amino acid sequence thereto.

The amino acid sequence of the enzyme pgl from E. coli is shown in SEQ ID NO:24, and the nucleotide sequence of the naturally-occurring gene encoding the enzyme pgl of SEQ ID NO:24 is shown in SEQ ID NO:22. The present inventors optimized the nucleotide sequence of the naturally-occurring gene of SEQ ID NO:22 so as to improve its expression and activity in the host microorganism. The resulting optimized nucleotide sequence encoding the enzyme pgl of SEQ ID NO:24 is shown in SEQ ID NO:23.

Specifically, the enzyme PGL may preferably have a polypeptide with an amino acid sequence with a homology (preferably identity) of 80% or more, particularly 85% or more, still particularly 90% or more, even still particularly 95% or more, or 96% or more, or 97% or more, or 99% or more, especially 100%, to the amino acid sequence shown in SEQ ID NO:24.

6-Phosphogluconate dehydrogenase (PGD) may be an enzyme comprising a polypeptide having the amino acid sequence shown in SEQ ID NO:27, or a polypeptide having a similar amino acid sequence thereto.

The amino acid sequence of the enzyme gnd from E. coli is shown in SEQ ID NO:27, and the nucleotide sequence of the naturally-occurring gene encoding the enzyme gnd of SEQ ID NO:27 is shown in SEQ ID NO:25. The present inventors optimized the nucleotide sequence of the naturally-occurring gene of SEQ ID NO:25 so as to improve its expression and activity in the host microorganism. The resulting optimized nucleotide sequence encoding the enzyme gnd of SEQ ID NO:27 is shown in SEQ ID NO:26.

Specifically, the enzyme PGD may preferably have a polypeptide with an amino acid sequence with a homology (preferably identity) of 80% or more, particularly 85% or more, still particularly 90% or more, even still particularly 95% or more, or 96% or more, or 97% or more, or 99% or more, especially 100%, to the amino acid sequence shown in SEQ ID NO:27.

Ribose-5-phosphate isomerase (RPI) may be an enzyme comprising a polypeptide having the amino acid sequence shown in SEQ ID NO:30 or SEQ ID NO:33, or a polypeptide having a similar amino acid sequence thereto.

The amino acid sequence of the enzyme rpiA from E. coli is shown in SEQ ID NO:30, and the nucleotide sequence of the naturally-occurring gene encoding the enzyme rpiA of SEQ ID NO:30 is shown in SEQ ID NO:28. The present inventors optimized the nucleotide sequence of the naturally-occurring gene of SEQ ID NO:28 so as to improve its expression and activity in the host microorganism. The resulting optimized nucleotide sequence encoding the enzyme rpiA of SEQ ID NO:30 is shown in SEQ ID NO:29.

The amino acid sequence of the enzyme rpiB from E. coli is shown in SEQ ID NO:33, and the nucleotide sequence of the naturally-occurring gene encoding the enzyme rpiB of SEQ ID NO:33 is shown in SEQ ID NO:31. The present inventors optimized the nucleotide sequence of the naturally-occurring gene of SEQ ID NO:31 so as to improve its expression and activity in the host microorganism. The resulting optimized nucleotide sequence encoding the enzyme rpiB of SEQ ID NO:33 is shown in SEQ ID NO:32.

Specifically, the enzyme RPI may preferably have a polypeptide with an amino acid sequence with a homology (preferably identity) of 80% or more, particularly 85% or more, still particularly 90% or more, even still particularly 95% or more, or 96% or more, or 97% or more, or 99% or more, especially 100%, to the amino acid sequence shown in SEQ ID NO:30 or SEQ ID NO:33.

Phosphoribosyl pyrophosphate synthase (PRS) may be an enzyme comprising a polypeptide having the amino acid sequence shown in SEQ ID NO:36, or a polypeptide having a similar amino acid sequence thereto.

The amino acid sequence of the enzyme prs from E. coli is shown in SEQ ID NO:36, and the nucleotide sequence of the naturally-occurring gene encoding the enzyme prs of SEQ ID NO:36 is shown in SEQ ID NO:34. The present inventors optimized the nucleotide sequence of the naturally-occurring gene of SEQ ID NO:34 so as to improve its expression and activity in the host microorganism. The resulting optimized nucleotide sequence encoding the enzyme prs of SEQ ID NO:36 is shown in SEQ TD NO:35.

Specifically, the enzyme PRS may preferably have a polypeptide with an amino acid sequence with a homology (preferably identity) of 80% or more, particularly 85% or more, still particularly 90% or more, even still particularly 95% or more, or 96% or more, or 97% or more, or 99% or more, especially 100%, to the amino acid sequence shown in SEQ ID NO:36.

The NMN synthase, niacin transporter, NAm derivative transporter, and/or PRPP synthesis-related enzymes (PGI, GPD, PGL, PGD, RPI, and PRS) can be derived from any source. In other words, each of these enzymes and transporters may be either a gene endogenous to the host organism or a gene derived from any gene exogenous to the host microorganism and artificially modified so as to be expressed in the host microorganism via genetic recombination or any other means.

The enzymes and transporters of the invention with improved activity as mentioned above, especially the NMN synthase of the invention, can be recovered from the host microorganism via general means such as extraction and isolation, and can preferably be used for other applications such as enzymatic reactions as appropriate.

### III. Vectors

This chapter deals with the vectors for expressing the enzymes involved in the production of NAm derivatives used in the present invention.

According to an aspect of the invention, a vector carrying a nucleic acid encoding the amino acid sequence(s) of the NMN synthase, niacin transporter, NAm derivative transporter, and/or PRPP synthases (PGI, GPD, PGL, PGD, RPI, and PRS) is produced, and used for introducing the enzyme(s)/transporter(s) into the host microorganism. There is no limitation to the combination of the enzyme(s)/transporter(s) to be carried by the vector. Each enzyme/transporter may be carried by a separate vector, or two or more of the enzyme(s)/transporter(s) may be carried together by a single vector.

An exemplary vector according to the present invention is a vector carrying a nucleic acid encoding the amino acid sequence of the NMN synthase (NAMPT) of the present invention as described above. Such vectors may be referred to as “the NMN synthase vector of the present invention” or as “the NAMPT vector of the present invention” as appropriate.

The NMN synthase vector of the present invention may preferably carry, as the nucleic acid encoding the NMN synthase of the present invention, a nucleic acid having a nucleotide sequence with an identity of 80% or more, particularly 85% or more, more particularly 90% or more, even more particularly 95% or more, or 96% or more, or 97% or more, or 99% or more, especially 100%, to the nucleotide sequence shown in SEQ ID NO:2 or SEQ ID NO:5.

Another exemplary vector according to the present invention is a vector carrying a nucleic acid encoding the amino acid sequence of the niacin transporter of the present invention as described above. Such vectors may be referred to as “the niacin transporter vector of the present invention” as appropriate.

The niacin transporter vector of the present invention may preferably carry, as the nucleic acid encoding the niacin transporter of the present invention, a nucleic acid having a nucleotide sequence with an identity of 80% or more, particularly 85% or more, more particularly 90% or more, even more particularly 95% or more, or 96% or more, or 97% or more, or 99% or more, especially 100%, to the nucleotide sequence shown in SEQ ID NO:8 or SEQ ID NO:11.

Still another exemplary vector according to the present invention is a vector carrying a nucleic acid encoding the amino acid sequence of the NAm derivative of the present invention as described above. Such vectors may be referred to as “the NAm derivative transporter vector of the present invention” as appropriate.

The NAm derivative transporter vector of the present invention may preferably carry, as the nucleic acid encoding the NAm derivative transporter of the present invention, a nucleic acid having a nucleotide sequence with an identity of 80% or more, particularly 85% or more, more particularly 90% or more, even more particularly 95% or more, or 96% or more, or 97% or more, or 99% or more, especially 100%, to the nucleotide sequence shown in SEQ ID NO:14.

Still another exemplary vector according to the present invention is a vector carrying a nucleic acid encoding the amino acid sequence(s) of one or more of the PRPP synthetases as described above, i.e., PGI, GPD, PGL, PGD, RPI, and PRS. Such vectors may be collectively referred to as “the PRPP synthase vectors of the present invention,” and each may also be referred to using the name of the enzyme corresponding to the nucleic acid to be carried, as, e.g., “the GPI enzyme vector of the present invention.”

Specifically, the GPI vector of the present invention may preferably carry, as the nucleic acid encoding the GPI of the present invention, a nucleic acid with a nucleotide sequence with an identity of 80% or more, particularly 85% or more, still particularly 90% or more, even still particularly 95% or more, or 96% or more, or 97% or more, or 99% or more, especially 100%, to the nucleotide sequence shown in SEQ ID NO:17.

The GPD vector of the present invention may preferably carry, as the nucleic acid encoding the GPD of the present invention, a nucleic acid with a nucleotide sequence with an identity of 80% or more, particularly 85% or more, still particularly 90% or more, even still particularly 95% or more, or 96% or more, or 97% or more, or 99% or more, especially 100%, to the nucleotide sequence shown in SEQ ID NO:20.

The PGL vector of the present invention may preferably carry, as the nucleic acid encoding the PGL of the present invention, a nucleic acid with a nucleotide sequence with an identity of 80% or more, particularly 85% or more, still particularly 90% or more, even still particularly 95% or more, or 96% or more, or 97% or more, or 99% or more, especially 100%, to the nucleotide sequence shown in SEQ ID NO:23.

The PGD vector of the present invention may preferably carry, as the nucleic acid encoding the PGD of the present invention, a nucleic acid with a nucleotide sequence with an identity of 80% or more, particularly 85% or more, still particularly 90% or more, even still particularly 95% or more, or 96% or more, or 97% or more, or 99% or more, especially 100%, to the nucleotide sequence shown in SEQ ID NO:26.

The RPI vector of the present invention may preferably carry, as the nucleic acid encoding the RPI of the present invention, a nucleic acid with a nucleotide sequence with an identity of 80% or more, particularly 85% or more, still particularly 90% or more, even still particularly 95% or more, or 96% or more, or 97% or more, or 99% or more, especially 100%, to the nucleotide sequence shown in SEQ ID NO:29 or SEQ ID NO:32.

The PRS vector of the present invention may preferably carry, as the nucleic acid encoding the PRS of the present invention, a nucleic acid with a nucleotide sequence with an identity of 80% or more, particularly 85% or more, still particularly 90% or more, even still particularly 95% or more, or 96% or more, or 97% or more, or 99% or more, especially 100%, to the nucleotide sequence shown in SEQ ID NO:35.

In the present disclosure, a vector carrying nucleic acids of two or more enzymes is referred to by the names of the enzyme linked with a slash. For example, the term “GPI/GPD/PGI/PGD/RPI/PRS vector” means a vector carrying nucleic acids encoding the amino acid sequences of GPI, GPD, PGL, PGD, RPI, and PRS.

Each vector mentioned herein may be in any form, as long as it has a nucleic acid region encoding an amino acid sequence of the corresponding enzyme (hereinafter referred to as the “coding region”). For example, it may be either a linear vector or a circular vector. Each DNA to be incorporated into the genome of the host cell may be either carried by a single vector or divided and carried by two or more vectors.

There is no limitation to the replication capacity of each vector mentioned herein. For example, each vector may be an autonomously replicable vector, i.e., a vector that exists outside the chromosomes of the host cell and replicates independently of the chromosome replication. Examples of such autonomously replicable vectors include plasmid vectors, extrachromosomal elements, minichromosomes, and artificial chromosomes. In this case, the vector may usually contain, in addition to the nucleic acid encoding the enzyme mentioned above, functional elements necessary for autonomous replication, such as a replication origin. Examples of replication origins that can be used in E. coli host cells include pUC replication origin, RSF replication origin, p15A replication origin, ColDF13 replication origin, ColE1 replication origin, pBR322 replication origin, pACYC replication origin, pSC101 replication origin, fl replication origin, M13 replication origin, BAC vector replication origin, PAC vector replication origin, cosmid vector replication origin, etc. Examples of replication origins that can be used in yeast host cells include the 2μ origins, ARS, etc.

Alternatively, each vector mentioned herein may not be capable of autonomous replication, and may be incorporated into the genome of the host cell when introduced into the host cell, and replicated together with the host genome. In this case, the nucleic acids to be incorporated into the host cell genome may be carried by a single vector or may be divided and carried by two or more vectors. Examples of such vectors lacking autonomous replication ability include virus vectors, phage vectors, cosmid vectors, and fosmid vectors.

Such vectors lacking autonomous replication ability may be configured to be precisely incorporated by homologous recombination into a desired position in a desired chromosome of the host cell. In this case, the nucleic acid to be incorporated into the genome of the host cell may be sandwiched between a pair of flanking sequences having complementary nucleotide sequences on both sides of the desired integration site. The length of each flanking sequence is not restricted, but may be, e.g., 50 bases or more, 100 bases or more, or 200 bases or more. Such recombination can also be achieved using various known recombinases, such as Red recombinase from lambda phage and RecE/RecT recombinase from Rac prophage.

Alternatively, such vectors lacking autonomous replication ability may be configured to be incorporated into the genome of the host cell by non-homologous recombination. In this case, the nucleic acids to be incorporated into the genome of the host cell may be sandwiched by the RB and LB sequences derived from the T-DNA of Agrobacterium, or by various known transposon sequences. Alternatively, the desired nucleic acid may be inserted into the genome of the host cell using genome editing technology.

In addition to the coding region of the enzyme and sequences for autonomous replication and/or for integration into the genome as mentioned above, each vector of the present invention may also contain one or more additional nucleic acid regions having other functions. Examples include regulatory sequences that control the expression of the coding region, selection marker genes, and multi-cloning sites.

Examples of regulatory sequences include promoters, ribosome binding sequences, enhancers, cis-elements, terminators, and the like. Such regulatory sequences may be selected and used based on, e.g., the type of the host cell to be used, the size of the enzyme, etc. Specific examples include, but are not limited to, the following.

Examples of promoters that can be used in E. coli host cells include: trp promoter, lac promoter, PL promoter, PR promoter, tac promoter, T7 promoter, and T5 promoter. Examples of promoters that can be used in yeast host cells include: gal1promoter, gal10 promoter, heat shock protein promoter, MFα1 promoter, PHO5 promoter, PGK promoter, GAP promoter, ADH promoter, and AOX1 promoter.

Any known ribosome-binding sequence for use in various host cells can be used so long as it allows mRNA transcribed from DNA to bind to ribosomes in the host cell when the biosynthesis of a protein is initiated.

Examples of terminators that can be used in E. coli host cells include T7 terminator, fd phage terminator, T4 terminator, the terminator of the tetracycline resistance gene, and the terminator of the E. coli trpA gene. Examples of terminators that can be used in yeast host cells include PGK1 terminator, CYC1 terminator, and DIT1 terminator.

The coding region of any of the enzymes mentioned above may be operably linked to such regulatory sequences (e.g., a promoter, a ribosome-binding sequence, and a terminator) in advance such that the enzyme can be expressed from the coding region under the control of these regulatory sequences.

Alternatively, the coding region of any of the enzymes mentioned above may be configured to be operably linked to such regulatory sequences (e.g., promoters, ribosome-binding sequences, and terminators) of the host cell or of the vector upon recombination such that the enzyme can be expressed from the coding region under the control of these regulatory sequences.

A selection marker gene may be used for confirming that the vector has been properly introduced into the host cell and (in the case of vectors lacking the ability of autonomous replication) incorporated into the genome. Any sequence can be selected as the selection marker gene depending on the type of the host cell to be used. Examples of selection markers that can be used in E. coli host cells include, although not limited to: Ampr, Tetr, Cmr, Kmr, Spcr, Smr, Hygr, Gmr, Rifr, Zeocinr, and Blasticidinr. Examples of selectable markers include, although not limited to: URA3, TRP1, SUP4, ADE2, HIS3, LEU2, LYS2, KANMX, AUR1-C, CYH2, CAN1, PDR4, and hphMX.

The selection marker gene may preferably be operably linked to regulatory sequences (e.g., a promoter, a ribosome-binding sequence, and a terminator) and constitute a cassette having the ability to be expressed autonomously, such that it can be expressed in the host cell as appropriate. The regulatory sequences for the expression of the selection marker gene may be prepared independently of the regulatory sequences for the expression of the enzyme(s) as described above, or the selection marker gene may share the same regulatory sequences with the enzyme(s) as described above.

After the host cells are transformed with the vector, the transformed cells are incubated under selection conditions that allow only cells expressing the selection marker to survive, in order to select cells in which the vector has been properly introduced and (in the case of vectors lacking the ability of autonomous replication) incorporated into the genome.

### IV. Recombinant Microorganism

This chapter deals with the recombinant microorganisms used in the present invention to produce NMN.

An aspect of the present invention relates to a recombinant microorganism expressing the NMN synthases, niacin transporters, NAm derivative transporters, and/or PRPP synthesis-related enzymes (GPI, GPD, PGL, PGD, RPI, and/or PRS) mentioned above. Such microorganisms may be referred to as “the recombinant microorganisms of the present invention.”

The recombinant microorganism of the present invention may be obtained by transforming a host microorganism with the vector of the present invention described above. The biological species of the recombinant microorganism and its host microorganism is not particularly limited, but may preferably be a bacterium or fungus. Examples of bacteria include, although not limited to, those belonging to the genera Escherichia, Staphylococcus, Bacillus, Pseudomonas, Proteus, Corynebacterium, and Actinomyces, among which those belonging to the genus Escherichia (e.g., E. coli) or Corynebacterium may preferably be used. Examples of fungi include, although not limited to, yeasts and filamentous fungi, of which yeasts are preferred. Examples of yeasts include those belonging to the genera Saccharomyces, Candida, Yarrowia, Pichia, and Kluyveromyces.

Some types of host microorganisms may have the ability to express endogenous enzymes corresponding to the NAMPT, the niacin transporter, the NAm derivative transporter, and/or the PRPP synthesis-related enzymes. In such cases, the endogenous NAMPT, niacin transporter, NAm derivative transporter, and/or PRPP synthesis-related enzymes may be used for the biosynthesis of NMN. However, even if the host microorganism is capable of expressing endogenous enzymes corresponding to NAMPT, niacin transporters, NAm derivative transporters, and/or PRPP synthesis-related enzymes, it is preferable to genetically modify the host microorganism to express these enzymes/transporters from the perspective of achieving higher expression of these enzymes/transporters and improved efficiency of final NAm derivative production.

Specifically, according to an aspect of the present invention, the recombinant microorganism may preferably be genetically modified to express at least the NMN synthase of the present invention, and may more preferably be genetically modified to also express the NAm derivative transporter and/or the niacin transporter of the present invention. In addition, the recombinant microorganism of the present invention may preferably be genetically modified to express at least any one of the PRPP synthesis-related enzymes, specifically GPI, GPD, PGL, PGD, RPI, and PRS, and may more preferably be genetically modified to express any two, three, four, or five, an even more preferably all, of GPI, GPD, PGL, PGD, RPI, and PRS.

Any offspring obtained by growing the recombinant microorganism of the present invention also fall under the scope of the recombinant microorganism of the present invention as long as they maintain the ability to express the NMN synthase, niacin transporter, NAm derivative transporter, and/or PRPP synthesis-related enzymes mentioned above.

The recombinant microorganism of the present invention may only have to be capable of expressing the NMN synthase, niacin transporter, NAm derivative transporter, and/or PRPP synthesis-related enzymes mentioned above. However, various modifications may be made thereto in consideration of the production efficiency of the desired NAm derivative.

An example of such modification is genetic recombination causing knockout or knockdown of any of various enzymes which otherwise may lead to a decrease in the production efficiency of the target NAm derivative.

For example, if the NAm derivative to be manufactured is NMN, the enzymes involved in the conversion of NMN to various other NAms mentioned above (specifically, nicotinamide/nicotinate mononucleotide adenylyltransferase (NMNAT), which converts NMN to NAD, nicotinamide nucleotide amidase (NANA), which converts NMN to nicotinic acid mononucleotide (NaMN), nicotinamide mononucleotide-5-nucleotidase (NMNN), which converts NMN to nicotinamide riboside (NR), etc.) are unnecessary; rather, the presence of these enzymes may lead to a decrease in the production efficiency of NMN. It may therefore be preferable to knock-out or knock-down the genes of these enzymes in order to prevent or reduce their expression and to prevent the synthesized NMN from being converted to such other NAm derivatives.

If the NAm derivative to be manufactured is a derivative other than NMN, such as NAD, NaMN, NR, etc., the gene of the enzyme that converts NMN into the desired NAm derivative may preferably be promoted (e.g., by means of external gene transfer, etc.), while the genes of the enzymes that convert NMN into the other derivative may preferably be knocked out or knocked down in order to prevent or reduce their expression, so as not for the synthesized NMN to be converted into other NAm derivatives than the desired NAm derivative. Alternatively, NMN produced by the microorganism of the present invention may further be converted into the desired NAm derivative via enzymatic or chemical reaction.

Various methods of knocking out or knocking down the genes of various enzymes via genetic recombination are known in the art.

Another example of modification is to carry out physical or chemical treatment, or both, on the cell surface of the recombinant microorganism, instead of or in conjunction with the recombinant expression of niacin transporters and/or NAm derivative transporters, etc., in order to facilitate the intracellular uptake of NAm, a reactant of the NMN synthesis reaction and also to facilitate the extracellular excretion of the produced NMN. Examples of physical treatments include, although not limited to, freezing, drying, and sonication of the microorganism. Examples of chemical treatments include, although not limited to, addition of surfactants such as Triton X-100, Triton X-114, NP-40, Tween-20, Tween-80, and CHAPS; addition of organic solvents such as alcohols and xylene; and Mn²⁺-restricted culture.

### V. Method of Producing NAm Derivatives

This chapter deals with the method of producing NAm derivatives using the recombinant microorganisms mentioned above.

An aspect of the present invention relates to a method for producing a NAm derivative, the method comprising supplying NAm to the recombinant microorganism of the present invention described above and then recovering the NAm derivative produced by the microorganism. This production method may be referred to as “the method of NAm derivative production of the present invention” as appropriate.

The following description will be made on various procedures and conditions of the method of NAm derivative production of the present invention, mainly with reference to an example where the NAm derivative is NMN and the host microorganism is E. coli. However, the method of NAm derivative production of the present invention is not limited to the one using the procedures and conditions of described below, but may be carried out with making various modifications thereto.

The method for feeding NAm to the recombinant microorganism of the present invention is not particularly limited. For example, NAm may be added directly to the culture medium in which the recombinant microorganism of the present invention is being cultured. However, in consideration of the efficiency of production and recovery of the resulting NAm derivative, the medium may preferably be removed via, e.g., centrifugation, and the resulting recombinant microorganism may preferably be added to a reaction solution which has a composition suitable for the reaction. The feeding of the recombinant microorganism may be carried out in bulk, continuously, or intermittently.

The composition of the reaction solution for the NAm derivative production is not limited. For example, it may only contain, as minimum components, the recombinant microorganism of the invention as well as NAm, which is the substrate for the NAm derivative synthesis reaction, in various media used for cultivation, such as an aqueous solution such as phosphate buffer or phosphate-buffered saline (PBS), or water. However, in order to promote the production of the NAm derivative, it may preferably also contain, as nutrient sources for the recombinant microorganism: organic carbon sources such as glucose, glycerol, fructose, starch, and blackstrap molass; and inorganic carbon sources such as carbonates, as well as phosphates such as potassium dihydrogen phosphate and dipotassium hydrogen phosphate as phosphorus components. Other components such as minerals, nitrogen sources, and ATP may be added as appropriate.

Any generally known synthetic or natural culture media can be used as various types of culture media, as long as they do not adversely affect the NAm derivative production.

The composition ratios of the reaction solution are not limited, but may be, for example, as follows:

Although the cell number of the recombinant microorganism is not limited, if the cell number is too low, the reaction may be carried out in such a diluted state that the reaction may not progress sufficiently, while if the cell number is too high, side reactions other than the desired NAm derivative production may occur. In terms of optical density (OD) measured at a wavelength appropriate for the microorganism, the cell number may preferably correspond to an OD of 1 or more, more preferably 5 or more, even more preferably 10 or more, and may preferably correspond to an OD of OD of 500 or less, more preferably 300 or less. In the case of E. coli, the OD may be measured at a wavelength of, e.g., 600 nm.

Although the concentration of NAm is not limited, if the concentration is too low, the amount of NAm taken up by the microorganism may be so low that the desired reaction may not proceed significantly, while if the concentration is too high, it may place a burden on the microorganism. Accordingly, the concentration may preferably be 10 mg/L or more, particularly 100 mg/L or more, more particularly 1000 mg/L or more, and may preferably be 300 g/L or less, particularly 250 g/L or less, more particularly 200 g/L or less.

Although the concentration of carbon source is not limited, if the concentration is too low, metabolism may not sufficiently proceed in the microorganism, while if the concentration is too high, it may place a burden on the microorganism. Accordingly, the concentration may preferably be 10 mg/L or more, particularly 50 mg/L or more, more particularly 100 mg/L or more, and may preferably be 300 g/L or less, particularly 250 g/L or less, more particularly 200 g/L or less.

Although the concentration of phosphorus component is not limited, if the concentration is too low, metabolism may not sufficiently proceed in the microorganism, while if the concentration is too high, it may place a burden on the microorganism. Accordingly, the concentration may preferably be 0.1 mmol/L or more, particularly 0.5 mmol/L or more, more particularly 1 mmol/L or more, and may preferably be 10 mol/L or less, particularly 5 mol/L or less, more particularly 1 mol/L or less.

Other components may also preferably be added in appropriate amounts according to the cell number of the microorganism and the composition ratios of the reaction solution to be used.

These components of the reaction solution may be mixed either simultaneously at once or sequentially in any order. For example, the components other than the recombinant microorganism of the invention and NAm may first be mixed to prepare a reaction solution of basic composition, and then the recombinant microorganism of the invention may be added to the reaction solution. When the recombinant microorganism of the invention starts cellular activity and growth, NAm, the reactant, may be added to start the synthesis reaction of the NAm derivative.

Part of the medium used for the pre-culture of the recombinant microorganism may remain in the reaction solution, so long as it does not interfere with the NAm derivative synthesis reaction.

The pH of the reaction solution may be adjusted as appropriate such that it becomes the optimal pH for the recombinant microorganism of the invention. However, from the viewpoint of the durability of the recombinant microorganism and the stability of the NAm derivative, the pH of the reaction solution may preferably be adjusted at pH 2 or more, especially 3 or more, and for the same reason, it may preferably be adjusted at pH 9 or less, especially 8 or less. The pH may be adjusted using a pH adjusting agent such as calcium carbonate, inorganic or organic acids, alkaline solutions, ammonia, and pH buffers.

The reaction conditions during the NAm derivative production are not limited, but may be, for example, as follows.

The temperature during the reaction may be adjusted as appropriate so long as it is optimal for the recombinant microorganism of the invention, but from the viewpoint of progressing the reaction, the temperature may preferably be 15° C. or more, particularly 20° C. or more, and from the viewpoint of durability of the recombinant microorganism and stability of the NAm derivative, the temperature may preferably be 50° C. or less, particularly 40° C. or less.

The pressure during the reaction is also not limited, but may typically be at ambient pressure.

The atmosphere during the reaction may be selected so as to be optimal for the recombinant microorganism of the present invention from, e.g., an ambient atmosphere, an aerobic atmosphere, a hypoxic atmosphere, or an anaerobic atmosphere.

During the reaction, the reaction solution may be shaken or stirred as appropriate.

Although the reaction time depends on, e.g., the type of the recombinant microorganism, the composition ratios of the reaction solution, and the reaction conditions, if the reaction time is too short, the NAm derivative production may not be sufficiently advanced, while if the reaction time is too long, the produced NAm derivatives may be converted or decomposed. For this reason, the reaction time may preferably be 0.1 hours or more, particularly 0.3 hours or more, more particularly 0.5 hours or more, and may preferably be 120 hours or less, particularly 96 hours or less, more particularly 72 hours or less.

The reaction method may be selected from any generally known methods depending on the microorganism used and the reaction conditions. Examples include batch type, continuous batch type, flow microreactor type, loop reactor type, and single-use type.

After the reaction, the NAm derivative produced by the recombinant microorganism of the present invention is recovered. Typically, the produced NAm derivative permeates the cell membrane of the recombinant microorganism of the invention and is secreted into the reaction solution, so the NAm derivative can be isolated and purified from the reaction solution.

Although the methods of isolation and purification are not particularly limited, general methods can be used such as removal of the bacteria, removal of impurities from the fermentation culture supernatant, purification and recovery of the target product. These processes may be used singly, but may preferably be used in combination of any two or more.

The process for removing the bacteria may be selected from any generally known methods. Specific examples include centrifugation, membrane separation, etc.

The process for removing impurities from the fermentation culture supernatant may be selected from any generally known methods. Specific examples include activated carbon treatment, filtration (specifically, including filtration by reverse osmosis membrane, nanofiltration membrane, microfiltration membrane, ultrafiltration membrane, microfiltration membrane, etc.) treatment, ion exchange resin, etc.

The process for purifying and recovering the target product may be selected from any generally known methods. Specific examples include affinity column chromatography, vacuum concentration, membrane concentration, lyophilization, solvent extraction, distillation, separation by column chromatography, separation by ion-exchange column, high-performance liquid chromatography (HPLC) method, and precipitation by recrystallization.

These processes can be used in combination. For example, isolation and purification may preferably be carried out by combining centrifugation, activated carbon treatment, ion exchange resin, nanofiltration membrane treatment, and recrystallization.

The pH during the isolation and purification is not particularly limited, but from the standpoint of the stability of the NAm derivative, the pH range may preferably be pH 2 or more, particularly 3 or more, and may preferably be pH 9 or less, particularly 8 or less. The pH may be adjusted via any method selected from generally known methods. Specific examples include pH adjusting agents such as calcium carbonate, inorganic or organic acids, alkaline solutions, ammonia, and pH buffers.

The temperature during the isolation and purification is not particularly limited, but the lower limit may preferably be 10° C. or more, particularly 15° C. or more, more particularly 20° C. or more. If the temperature is lower than the lower limit mentioned above, the NAm derivative may precipitate and make it difficult to carry out the desired isolation and purification process. On the other hand, the upper limit may preferably be 50° C. or less, particularly 45° C. or less, more particularly 40° C. or less. If the temperature is higher than the upper limit mentioned above, the NAm derivative may decompose. However, when heating or cooling is performed during various isolation and purification processes, the temperature may temporarily deviate from the aforementioned suitable range.

The pH during recrystallization in the isolation and purification process is not particularly limited, but from the viewpoint of stability and ease of crystallization of the NAm derivative, the pH may preferably be adjusted to within the range of from 2 to 5, more preferably within the range of from 2 to 4. The acid to be used for pH adjustment is not limited, but may be selected from, e.g., hydrochloric acid, phosphoric acid, tartaric acid, malic acid, benzoic acid, acetic acid, succinic acid, and gluconic acid. Among them, hydrochloric acid may be most preferred.

If the NAm derivative remains in the recombinant microorganism cells, the cell membrane may be disrupted by methods such as homogenization, lysozyme, sonication, freeze-thawing, French pressing, or any other chemical, mechanical, or physical cell disruption method to excretion the NAm derivative into the reaction solution before the cells are subject to the isolation and purification of the NAm derivative.

### VI. Others

Although the invention has been explained in detail with reference to specific embodiments so far, the present invention should not be limited to the above-mentioned embodiments in any way, but may be implemented in any form so long as it does not deviate from the gist of the present invention.

### EXAMPLES

The invention will be described in further detail with reference to the Examples indicated below. However, the present invention should also not be limited to these Examples in any way, but may be implemented in any form so long as it does not deviate from the gist of the present invention.

### I. Measurement Conditions

Quantification of NMN:

### Instrument: LCMS-2020 (Shimadzu Corporation)

### Detector: 254 nm

Column: TSK gel Amide-80, 3 μm, 4.6 mm×50 mm
Column temperature: 30° C.
Injection volume: 5 μL
Mobile phases: A: 0.1% formic acid in water
B: Acetonitrile/methanol (75/25) containing 0.1% formic acid

### <Condition>

Flow rate: 1 mL/min constant
Mobile phase ratio: 0->2 min (B=98% constant),
2->6 min (B=98->60%),
6->8 min (B=60->45%),
8->12 min (B=45->60%),
12->15 min (B=60->98%)
   Measurement time: 15.1 min.
   Quantification method: NMN standard samples were prepared with water at 0 g/L, 0.01 g/L, 0.05 g/L, 0.1 g/L, 0.25 g/L, 1 g/L, and 2.5 g/L. NMN area values obtained by measuring these samples were used to prepare a calibration curve. The NMN area value obtained by measuring each sample was used to quantify the NMN amount based on the calibration curve. Values less than 0.01 g/L was considered to be below the quantification limit.

Concentration of Bacterial Cells (OD):

### Instrument: UVmini-1240 (Shimadzu Corporation)

Measurement wavelength: 600 nm
Cell: 1.5 mL disposable cell (Material: PS)
Measurement method: A bacterial solution was diluted with water such that the measurement value was within the range of from 0.05 to 1.0. A cell containing 1 mL of culture medium diluted at the same ratio was set to the instrument to determine the zero point, and then a cell containing 1 mL of the prepared sample solution was set to the instrument and measured for OD₆₀₀.

### II. Materials

E. coli:

BL21 (DE3) strain (NEB)

Vectors:

pRSFDuet-1 (Novagen)
pCDFDuet-1 (Novagen)
pACYCDuet-1 (Novagen)

Synthetic Genes:

Chitinophaga pinensis-derived NAMPT (nicotinamide phosphoribosyl transferase: NMN synthetase)
Sphingopyxis sp. C-1-derived NAMPT
Homo sapiens-derived NAMPT
Burkholderia cenocepacia-derived niaP (niacin transporter)
Streptococcus pneumoniae TIGR4-derived niaX (niacin transporter)
Bacillus mycoides-derived pnuC (nicotinamide mononucleotide transporter)
E. coli K12-derived pgi (phosphoglucose isomerase)
E. coli K12-derived zwf (glucose 6-phosphate dehydrogenase)
E. coli K12-derived pgl (6-phosphogluconolactonase)
E. coli K12-derived gnd (6-phosphogluconate dehydrogenase)
E. coli K12-derived rpiA (ribose-5-phosphate isomerase)
E. coli K12-derived rpiB (ribose-5-phosphate isomerase)
E. coli K12-derived prs (phosphoribosyl pyrophosphate synthase)

SEQ ID NO:39 indicates the amino acid sequence of NAMPT derived from Homo sapiens, and SEQ ID NO:37 indicates the nucleotide sequence of the naturally-occurring gene encoding the NAMPT of SEQ ID NO:39. SEQ ID NO:37 indicates the nucleotide sequence of the naturally-occurring gene encoding the NAMPT of SEQ ID NO:39. SEQ ID NO:38 indicates the nucleotide sequence encoding the NAMPT of SEQ ID NO:39, which was optimized by the present inventors based on the sequence of the naturally-occurring gene such that its expression and activity were improved in the host microorganism.

Medium Components and Substrate Components:

### D-glucose (Nacalai Tesque Co., Ltd.)

### Nicotinamide (Tokyo Chemical Industry Co., Ltd.)

### PBS (Nippon Gene Co., Ltd.)

Phosphate buffer: prepared by mixing 1 M potassium dihydrogen phosphate (Nacalai Tesque Co., Ltd.) and 1 M dipotassium hydrogen phosphate (Nacalai Tesque Co., Ltd.) to adjust the pH at 6.2, followed by sterilization via autoclaving.
LB medium: prepared by mixing sodium chloride (Nacalai Tesque Co., Ltd.) 10 g/L, tryptone (Nacalai Tesque Co., Ltd.) 10 g/L, and dried yeast extract (Nacalai Tesque Co., Ltd.) 5 g/L, followed by sterilization via autoclaving.
M9 medium: prepared by mixing 48 mM disodium hydrogen phosphate (Nacalai Tesque Co., Ltd.), 22 mM potassium dihydrogen phosphate (Nacalai Tesque Co., Ltd.), 19 mM ammonium chloride (Nacalai Tesque Co., Ltd.), and 8.6 mM sodium chloride (Nacalai Tesque Co., Ltd.), followed by sterilization via autoclaving.

### III. Construction of Vectors

Construction of pRSF-NAMPT CP:

The synthetic gene of NAMPT derived from Chitinophaga pinensis (SEQ ID NO:5), codon-optimized for expression in E. coli, was amplified via PCR using the following primer pair, each containing homologous regions that can be linked to pRSFDuet-1 digested with restriction enzymes NcoI and EcoRI, respectively. The amplified product was then linked to pRSFDuet-1, which had been digested with restriction enzymes NcoI and EcoRI, using the In-Fusion cloning method to thereby produce pRSF-NAMPT CP.

(Primer pair for NAMPT CP)
*Forward (SEQ ID NO: 40):
AGGAGATATACCATGACCAAAGAAAACCTGATTCTGCTGGCAGATGCA
*Reverse (SEQ ID NO: 41):
GCTCGAATTCGGATCTTAGATGGTTGCGTTTTTACGGATCTGCTCAAA

Construction of pRSF-NAMPT SSC

The synthetic gene of NAMPT derived from Sphingopyxis sp. C-1 (SEQ ID NO:2), codon-optimized for expression in E. coli, was amplified via PCR using the following primer pair, each containing homologous regions that can be linked to pRSFDuet-1 digested with restriction enzymes NcoI and EcoRI, respectively. The amplified product was then linked to pRSFDuet-1, which had been digested with restriction enzymes NcoI and EcoRI, using the In-Fusion cloning method to thereby produce pRSF-NAMPT SSC.

(Primer pair for NAMPT SSC)
*Forward (SEQ ID NO: 42):
AGGAGATATACCATGAAGAATCTGATTCTGGCCACCGATAGCTATAAA
*Reverse (SEQ ID NO: 43):
GCTCGAATTCGGATCTTAACGACCTTCGCTACGTTTACGAACTGCATC

Construction of pRSF-NAMPT HS

The synthetic gene of NAMPT derived from Homo sapiens (SEQ ID NO:38), codon-optimized for expression in E. coli, was amplified via PCR using the following primer pair, each containing homologous regions that can be linked to pRSFDuet-1 digested with restriction enzymes NcoI and EcoRI, respectively. The amplified product was then linked to pRSFDuet-1, which had been digested with restriction enzymes NcoI and EcoRI, using the In-Fusion cloning method to thereby produce pRSF-NAMPT HS.

(Primer pair for NAMPT HS)
*Forward (SEQ ID NO: 44):
AGGAGATATACCATGAATCCGGCAGCAGAAGCCGAATTTAACATTCTG
*Reverse (SEQ ID NO: 45):
GCTCGAATTCGGATCTTAATGATGTGCTGCTTCCAGTTCAATGTTCAG

Construction of pCDF-prs->pgi:

The synthetic genes for pgi, zwf, pgl, gnd, rpiA, rpiB, and prs derived from E. coli K12 (SEQ ID NOs: 17, 20, 23, 26, 29, 32, and 35, respectively), codon-optimized for expression in E. coli, were amplified by PCR using the following primer pairs, each containing homologous regions that can be linked to pRSFDuet-1 and, except for prs, the same RBS regions as that of pCDFDuet-1. First, the fragments of prs, rpiB, rpiA, and gnd were linked to pCDFDuet-1, which had been digested with restriction enzymes NcoI and SacI, using the Gibson Assembly system. The resulting vector was then digested with the restriction enzyme SacI, and linked with the remaining fragments of pgl, zwf, and pgi, using the Gibson Assembly system to produce pCDF-prs->pgi.

(Primer pair for pgi)
*Forward (SEQ ID NO: 46):
CGTGATGGTCGTAGCTGGAATGAATTTGAATAAAAGGAGATATACCATGAA
GAACATTAATCCGACACAG
*Reverse (SEQ ID NO: 47):
ACTTAAGCATTATGCGGCCGCAAGCTTGTCGACCTGCAGGCGCGCCGTTAA
CCACGCCAGGCTTTATAAC
(Primer pair for zwf)
*Forward (SEQ ID NO: 48):
GTCAGGGTCCGATGTGGGTTGTTGTTAATGCACATTAAAAGGAGATATACC
ATGGCAGTTACCCAGACCG
*Reverse (SEQ ID NO: 49):
TTATTCAAATTCATTCCAGCTACG
(Primer pair for pgl)
*Forward (SEQ ID NO: 50):
AGAAGGTGtGTTTCATACAGAATGGCTGGACTAAAAGGAGATATACCATGA
AACAGACCGTGTATATTGC
*Reverse (SEQ ID NO: 51):
TTAATGTGCATTAACAACAACCC
(Primer pair for gnd)
*Forward (SEQ ID NO: 52):
TGGTACACCGGATGGTGTTAAAACCATTGTGAAATAAAAGGAGATATACCA
TGAGCAAACAGCAGATTGG
*Reverse (SEQ ID NO: 53):
CATTATGCGGCCGCAAGCTTGTCGACCTGCAGGCGCGCCGAGCTCTTAGTC
CAGCCATTCTGTATGAAAC
(Primer pair for rpiA)
*Forward (SEQ ID NO: 54):
CAATTACCGCAATTGAACAGCGTCGCAATTAAAAGGAGATATACCATGACC
CAGGATGAACTGAAAAAAG
*Reverse (SEQ ID NO: 55):
TTATTTCACAATGGTTTTAACACCATC
(Primer pair for rpiB)
*Forward (SEQ ID NO: 56):
AATGAAGAAAGCATTAGCGCCATGTTTGAACATTAAAAGGAGATATACCAT
GAAAAAAATCGCCTTTGGC
*Reverse (SEQ ID NO: 57):
TTAATTGCGACGCTGTTC
(Primer pair for prs)
*Forward (SEQ ID NO: 58):
ATTCCCCTGTAGAAATAATTTTGTTTAACTTTAATAAGGAGATATACCGTG
CCGGATATGAAACTGTTTG
*Reverse (SEQ ID NO: 59):
TTAATGTTCAAACATGGCGC

Construction of pCDF-pgi->prs:

The synthetic genes pgi, zwf, pgl, gnd, rpiA, rpiB, and prs derived from E. coli K12 (SEQ ID NOs: 17, 20, 23, 26, 29, 32, and 35, respectively), codon-optimized for expression in E. coli, were amplified by PCR using the following primer pairs, each containing homologous regions that can be linked to pRSFDuet-1 and, except for pgi, the same RBS regions as that of pCDFDuet-1. First, the fragments of pgi, zwf, pgl, and gnd were linked to pCDFDuet-1, which had been digested with restriction enzymes NcoI and SacI, using the Gibson Assembly system. The resulting vector was then digested with the restriction enzyme SacI, and linked with the remaining fragments of rpiA, rpiB, and prs, using the Gibson Assembly system to produce pCDF-pgi->prs.

(Primer pair for pgi)
*Forward (SEQ ID NO: 60):
TCCCCTGTAGAAATAATTTTGTTTAACTTTAATAAGGAGATATACCATGAA
GAACATTAATCCGACACAG
*Reverse (SEQ ID NO: 61):
TTAACCACGCCAGGCTTTATAAC
(Primer pair for zwf)
*Forward (SEQ ID NO: 62):
ATGGTCTGATTAATCGTTATAAAGCCTGGCGTGGTTAAAAGGAGATATACC
ATGGCAGTTACCCAGACCG
*Reverse (SEQ ID NO: 63):
TTATTCAAATTCATTCCAGCTACG
(Primer pair for pgl)
*Forward (SEQ ID NO: 64):
CCGTGATGGTCGTAGCTGGAATGAATTTGAATAAAAGGAGATATACCATGA
AACAGACCGTGTATATTGC
*Reverse (SEQ ID NO: 65):
TTAATGTGCATTAACAACAACCC
(Primer pair for gnd)
*Forward (SEQ ID NO: 66):
TCAGGGTCCGATGTGGGTTGTTGTTAATGCACATTAAAAGGAGATATACCA
TGAGCAAACAGCAGATTGG
*Reverse (SEQ ID NO: 67):
CATTATGCGGCCGCAAGCTTGTCGACCTGCAGGCGCGCCGAGCTCTTAGTC
CAGCCATTCTGTATGAAAC
(Primer pair for rpiA)
*Forward (SEQ ID NO: 68):
AAGGTGTGTTTCATACAGAATGGCTGGACTAAAAGGAGATATACCATGACC
CAGGATGAACTGAAAAAAG
*Reverse (SEQ ID NO: 69):
TTATTTCACAATGGTTTTAACACCATC
(Primer pair for rpiB)
*Forward (SEQ ID NO: 70):
GGTACACCGGATGGTGTTAAAACCATTGTGAAATAAAAGGAGATATACCAT
GAAAAAAATCGCCTTTGGC
*Reverse (SEQ ID NO: 71):
TTAATTGCGACGCTGTTC
(Primer pair for prs)
*Forward (SEQ ID NO: 72):
AAGCAATTACCGCAATTGAACAGCGTCGCAATTAAAAGGAGATATACCGTG
CCGGATATGAAACTGTTTG
*Reverse (SEQ ID NO: 73):
TCGACTTAAGCATTATGCGGCCGCAAGCTTGTCGACCTGCAGGCGCGCCGT
TAATGTTCAAACATGGCGC

Construction of pACYC-pgi->prs:

The synthetic genes pgi, zwf, pgl, gnd, rpiA, rpiB, and prs derived from E. coli K12 (SEQ ID NOs: 17, 20, 23, 26, 29, 32, and 35, respectively), codon-optimized for expression in E. coli, were amplified by PCR using the following primer pairs, each containing homologous regions that can be linked to pRSFDuet-1 and, except for pgi, the same RBS regions as that of pACYCDuet-1. First, the fragments of pgi, zwf, pgl, and gnd were linked to pACYCDuet-1, which had been digested with restriction enzymes NcoI and SacI, using the Gibson Assembly system. The resulting vector was then digested with the restriction enzyme SacI, and linked with the remaining fragments of rpiA, rpiB, and prs, using the Gibson Assembly system to produce pACYC-pgi->prs.

(Primer Pair for pgi)

Forward (SEQ ID NO:60: same as above)

Reverse (SEQ ID NO:61: same as above)

(Primer Pair for zwt)

Forward (SEQ ID NO:62: same as above)

Reverse (SEQ ID NO:63: same as above)

(Primer Pair for pgl)

Forward (SEQ ID NO:64: same as above)

Reverse (SEQ ID NO:65: same as above)

(Primer Pair for gnd)

Forward (SEQ ID NO:66: same as above)

Reverse (SEQ ID NO:67: same as above)

(Primer Pair for rpiA)

Forward (SEQ ID NO:68: same as above)

Reverse (SEQ ID NO:69: same as above)

(Primer Pair for rpiB)

Forward (SEQ ID NO:70: same as above)

Reverse (SEQ ID NO:71: same as above)

(Primer Pair for prs)

Forward (SEQ ID NO:72: same as above)

Reverse (SEQ ID NO:73: same as above)

Construction of pACYC-prs->pgi:

The synthetic genes pgi, zwf, pgl, gnd, rpiA, rpiB, and prs derived from E. coli K12 (SEQ ID NOs: 17, 20, 23, 26, 29, 32, and 35, respectively), codon-optimized for expression in E. coli, were amplified by PCR using the following primer pairs, each containing homologous regions that can be linked to pACYCDuet-1 and, except for prs, the same RBS regions as that of pACYCDuet-1. First, the fragments of prs, rpiB, rpiA, and gnd were linked to pACYCDuet-1, which had been digested with restriction enzymes NcoI and SacI, using the Gibson Assembly system. The resulting vector was then digested with the restriction enzyme SacI, and linked with the remaining fragments of pgl, zwf, and pgi, using the Gibson Assembly system to produce pACYC-prs->pgi.

(Primer Pair for pgi)

Forward (SEQ ID NO:46: same as above)

Reverse (SEQ ID NO:47: same as above)

(Primer Pair for zwf)

Forward (SEQ ID NO:48: same as above)

Reverse (SEQ ID NO:49: same as above)

(Primer Pair for pgl)

Forward (SEQ ID NO:50: same as above)

Reverse (SEQ ID NO:51: same as above)

(Primer Pair for gnd)

Forward (SEQ ID NO:52: same as above)

Reverse (SEQ ID NO:53: same as above)

(Primer Pair for rpiA)

Forward (SEQ ID NO:54: same as above)

Reverse (SEQ ID NO:55: same as above)

(Primer Pair for rpiB)

Forward (SEQ ID NO:56: same as above)

Reverse (SEQ ID NO:57: same as above)

(Primer Pair for prs)

Forward (SEQ ID NO:58: same as above)

Reverse (SEQ ID NO:59: same as above)

Construction of pACYC-niaP BC:

The synthetic gene of niaP derived from Burkholderia cenocepacia (SEQ ID NO: 8), codon-optimized for expression in E. coli, was amplified via PCR using the following primer pair, each containing homologous regions that can be linked to pACYCDuet1 digested with restriction enzymes NcoI and EcoRI, respectively. The amplified product was then linked to pACYCDuet-1, which had been digested with restriction enzymes NcoI and EcoRI, using the In-Fusion cloning method to thereby produce pACYC-niaP BC.

(Primer pair for niaP BC)
*Forward (SEQ ID NO: 74):
AGGAGATATACCATGCCTGCAGCAACCGCACC
*Reverse (SEQ ID NO: 75):
GCTCGAATTCGGATCTTAGCTTGCTTTATCTGCTGCTGTTGCCGGATAAC

Construction of pACYC-niaX SPT:

The synthetic gene of niaX derived from Streptococcus pneumoniae TIGR4 (SEQ ID NO: 11), codon-optimized for expression in E. coli, was amplified via PCR using the following primer pair, each containing homologous regions that can be linked to pACYCDuet1 digested with restriction enzymes NcoI and EcoRI, respectively. The amplified product was then linked to pACYCDuet-1, which had been digested with restriction enzymes NcoI and EcoRI, using the In-Fusion cloning method to thereby produce pACYC-niaX SPT.

(Primer pair for niaX SPT)
*Forward (SEQ ID NO: 76):
AGGAGATATACCTTGAGCGGTCTGCTGTATCACACCAGCGTTTATGCAG
*Reverse (SEQ ID NO: 77):
GCTCGAATTCGGATCTTAGCGACGTTTACGCAGAACTTTATAAACTGCC

Construction of pACYC-pnuC BM:

The synthetic gene of pnuC derived from Bacillus mycoides TIGR4 (SEQ ID NO: 14), codon-optimized for expression in E. coli, was amplified via PCR using the following primer pair, each containing homologous regions that can be linked to pACYCDuet1 digested with restriction enzymes NcoI and EcoRI, respectively. The amplified product was then linked to pACYCDuet-1, which had been digested with restriction enzymes NcoI and EcoRI, using the In-Fusion cloning method to thereby produce pACYC-pnuC BM.

(Primer pair for pnuC BM)
*Forward (SEQ ID NO: 78):
AGGAGATATACCATGGTTCGTAGTCCGCTGTTTCTGCTGATTAGCAGC
*Reverse (SEQ ID NO: 79):
GCTCGAATTCGGATCTTAGATGTAGTTGTTCACGCGTTCACGTTCTTTATG

Construction of pRSF-NAMPT CP+pnuC BM:

The synthetic gene of pnuC derived from Bacillus mycoides TIGR4 (SEQ ID NO: 14), codon-optimized for expression in E. coli, was amplified via PCR using the following primer pair, each containing homologous regions that can be linked to pRSF-NAMPT CP digested with restriction enzymes NcoI and EcoRI, respectively. The amplified product was then linked to pRSF-NAMPT CP, which had been digested with restriction enzymes NcoI and EcoRI, using the In-Fusion cloning method to thereby produce pRSF-NAMPT CP+pnuC BM.

(Primer pair for pnuC BM part2)
*Forward (SEQ ID NO: 80):
TATTAGTTAAGTATAAGAAGGAGATATACAATGGTTCGTAGTCCGCTGTTT
CTGCTGATTAGCAGC
*Reverse (SEQ ID NO: 81):
ATGCTAGTTATTGCTCAGCGGTGGCAGCAGTTAGATGTAGTTGTTCACGCG
TTCACGTTCTTTATG

Construction of CDF-pgi->prs+niP BC:

The synthetic gene of niaP derived from Burkholderia cenocepacia (SEQ ID NO: 8), codon-optimized for expression in E. coli, was amplified via PCR using the following primer pair, each containing homologous regions that can be linked to pCDF-pgi->prs digested with restriction enzymes BglII and AvrII, respectively. The amplified product was then linked to pCDF-pgi->prs, which had been digested with restriction enzymes BglII and AvrII, using the In-Fusion cloning method to thereby produce pCDF-pgi->prs+pnuC BC.

(Primer pair for niaP BC part2)
*Forward (SEQ ID NO: 82):
TATTAGTTAAGTATAAGAAGGAGATATACAATGCCTGCAGCAACCGCACC
*Reverse (SEQ ID NO: 83):
ATGCTAGTTATTGCTCAGCGGTGGCAGCAGTTAGCTTGCTTTATCTGCTGC
TGTTGCCGGATAAC

Construction of pRSF-NAMPT HS+pnuC BM:

The synthetic gene of pnuC derived from Bacillus mycoides (SEQ ID NO: 14), codon-optimized for expression in E. coli, was amplified via PCR using the following primer pair, each containing homologous regions that can be linked to pRSF-NAMPT HS digested with restriction enzymes BglII and AvrII, respectively. The amplified product was then linked to pRSF-NAMPT HS, which had been digested with restriction enzymes BglII and AvrII, using the In-Fusion cloning method to thereby produce pRSF-NAMPT HS+pnuC BM.

(Primer Pair for pnuC BM Part2)

Forward (SEQ ID NO:80: same as above)

Reverse (SEQ ID NO:81: same as above)

### IV. Establishment of Strains for Production

### Establishment of BL21/pRSF-NAMPT CP Strain (Example 1)

The pRSF-NAMPT CP was introduced into the BL21 (DE3) strain via the heat shock method to establish BL21/pRSF-NAMPT CP strain.

Establishment of BL21/pRSF-NAMPT SSC Strain:

The pRSF-NAMPT SSC was introduced into the BL21 (DE3) strain via the heat shock method to establish BL21/pRSF-NAMPT SSC strain.

### Establishment of BL21/pRSF-NAMPT CP/pCDF-prs->pgi Strain (Examples 2 and 7)

The pRSF-NAMPT CP and the pCDF-prs->pgi were introduced into the BL21 (DE3) strain via the heat shock method to establish BL21/pRSF-NAMPT CP/pCDF-prs->pgi strain.

### Establishment of BL21/pRSF-NAMPT CP/pCDF-prs->pgi/pACYC-pgi->prs Strain (Example 3)

The pRSF-NAMPT CP, the pCDF-prs->pgi, and the pACYC-pgi->prs were introduced into the BL21 (DE3) strain via the heat shock method to establish BL21/pRSF-NAMPT CP/pCDF-prs->pgi/pACYC-pgi->prs strain.

### Establishment of BL21/pRSF-NAMPT CP/pCDF-prs->pgi/pACYC-niaP BC Strain (Examples 4 and 8)

The pRSF-NAMPT CP, the pCDF-prs->pgi, and the pACYC-niaP BC were introduced into the BL21 (DE3) strain via the heat shock method to establish BL21/pRSF-NAMPT CP/pCDF-prs->pgi/pACYC-niaP BC strain.

### Establishment of BL21/pRSF-NAMPT CP/pCDF-prs->pgi/pACYC-niaX SPT Strain (Examples 5 and 9)

The pRSF-NAMPT CP, the pCDF-prs->pgi, and the pACYC-niaX SPT were introduced into the BL21 (DE3) strain via the heat shock method to establish BL21/pRSF-NAMPT CP/pCDF-prs->pgi/pACYC-niaX SPT strain.

### Establishment of BL21/pRSF-NAMPT CP/pCDF-prs->pgi/pACYC-pnuC BM Strain (Examples 6 and 10)

The pRSF-NAMPT CP, the pCDF-prs->pgi, and the pACYC-pnuC BM were introduced into the BL21 (DE3) strain via the heat shock method to establish BL21/pRSF-NAMPT CP/pCDF-prs->pgi/pACYC-pnuC BM strain.

### Establishment of BL21/pRSF-NAMPT CP+pnuC BM/pCDF-pgi->prs+niaP BC/pACYC-prs->pgi Strain (Example 11)

The pRSF-NAMPT CP+pnuC BM, the pCDF-pgi->prs+niaP BC, and the pACYC-prs->pgi were introduced into the BL21 (DE3) strain via the heat shock method to establish BL21/pRSF-NAMPT CP+pnuC BM/pCDF-pgi->prs+niaP BC/pACYC-prs->pgi strain.

### Establishment of BL21/pRSF-NAMPT HS Strain (Comparative Example 2)

The pRSF-NAMPT HS was introduced into the BL21 (DE3) strain via the heat shock method to establish BL21/pRSF-NAMPT HS strain.

### Establishment of BL21/pRSF-NAMPT HS+pnuC BM/pCDF-pgi->prs+niaP BC/pACYC-prs->pgi Strain (Comparative Example 3)

The pRSF-NAMPT HS+pnuC BM, the pCDF-pgi->prs+niaP BC, and the pACYC-prs->pgi were introduced into the BL21 (DE3) strain via the heat shock method to establish BL21/pRSF-NAMPT HS+pnuC BM/pCDF-pgi->prs+niaP BC/pACYC-prs->pgi strain.

### [V-1. Production of Nicotinamide Mononucleotide (NMN) 1]

### Example 1 (NMN Production Using the BL21/pRSF-NAMPT CP Strain)

The BL21/pRSF-NAMPT CP strain was inoculated into a test tube containing 5 ml of LB medium and incubated at 37° C. with 200 rpm for 12 hours. The culture was then inoculated into a 500 ml conical flask containing 200 ml of LB medium to achieve an OD₆₀₀ of 0.03, and incubated at 37° C. with 200 rpm. When the OD₆₀₀ reached 0.4, isopropyl-β-thiogalactopyranoside (Nakalai Tesque Co. Ltd.) was added to achieve a final concentration of 0.1 mM, and incubated at 25° C. with 200 rpm for 16 hours. The culture was then transferred to a 50-mL conical tube and centrifuged at 3000 g for 5 minutes to collect the bacterial cells. 1×PBS was added to the tube containing the recovered cells for washing, and the bacterial cells were collected by centrifugation at 3000 g for 5 minutes. This procedure was repeated twice. The collected bacteria were suspended in LB medium to achieve an OD₆₀₀ of 10, and 10 mL of the suspension was transferred into a 100-mL conical flask, to which 1 g/L of nicotinamide, 0.4 g/L of D-glucose, and 0.005 mol/L of phosphate buffer (pH 6.2) were added, and the reaction was allowed to run at 30° C. with 200 rpm. After 2 hours, the reaction liquid was collected, frozen at −30° C., thawed, and centrifuged at 12,000 rpm for 3 minutes to collect the supernatant. The collected liquid was subjected to HPLC analysis, which revealed that the amount of NMN was 0.03 g/L.

### Example 2 (NMN Production Using the BL21/pRSF-NAMPT CP/pCDF-prs->pgi Strain)

The reaction procedure was carried out in the same manner as in Example 1 except that the BL21/pRSF-NAMPT CP strain was changed to the BL21/pRSF-NAMPT CP/pCDF-prs->pgi strain. The collected liquid was subjected to HPLC analysis, which showed that the amount of NMN was 0.18 g/L.

### Example 3 (NMN Production Using the BL21/pRSF-NAMPT CP/pCDF-prs->pgi/pACYC-pgi->prs Strain)

The reaction procedure was carried out in the same manner as in Example 1 except that the BL21/pRSF-NAMPT CP strain was changed to the BL21/pRSF-NAMPT CP/pCDF-prs->pgi/pACYC-pgi->prs strain. The collected liquid was subjected to HPLC analysis, which showed that the amount of NMN was 0.22 g/L.

### Example 4 (NMN Production Using the BL21/pRSF-NAMPT CP/pCDF-prs->pgi/pACYC-niaP BC Strain)

The reaction procedure was carried out in the same manner as in Example 1 except that the BL21/pRSF-NAMPT CP strain was changed to the BL21/pRSF-NAMPT CP/pCDF-prs->pgi/pACYC-niaP BC strain. The collected liquid was subjected to HPLC analysis, which showed that the amount of NMN was 0.21 g/L.

### Example 5 (NMN Production Using the BL21/pRSF-NAMPT CP/pCDF-prs->pgi/pACYC-niaX SPT Strain)

The reaction procedure was carried out in the same manner as in Example 1 except that the BL21/pRSF-NAMPT CP strain was changed to the BL21/pRSF-NAMPT CP/pCDF-prs->pgi/pACYC-niaX SPT strain. The collected liquid was subjected to HPLC analysis, which showed that the amount of NMN was 0.23 g/L.

### Example 6 (NMN Production Using the BL21/pRSF-NAMPT CP/pCDF-prs->pgi/pACYC-pnuC BM Strain)

The reaction procedure was carried out in the same manner as in Example 1 except that the BL21/pRSF-NAMPT CP strain was changed to the BL21/pRSF-NAMPT CP/pCDF-prs->pgi/pACYC-pnuC BM strain. The collected liquid was subjected to HPLC analysis, which showed that the amount of NMN was 0.36 g/L.

### Example 7 (NMN Production Using the BL21/pRSF-NAMPT CP/pCDF-prs->pgi Strain)

The reaction procedure was carried out in the same manner as in Example 2 except that the nicotinamide amount was changed from 1 g/L to 2 g/L, the D-glucose amount from 0.4 g/L to 1.0 g/L, and the phosphate buffer (pH 6.2) amount from 0.005 mol/L to 0.01 mol/L. The collected liquid was subjected to HPLC analysis, which showed that the amount of NMN was 0.20 g/L.

### Example 8 (NMN Production Using the BL21/pRSF-NAMPT CP/pCDF-prs->pgi/pACYC-niaP BC Strain)

The reaction procedure was carried out in the same manner as in Example 7 except that the BL21/pRSF-NAMPT CP/pCDF-prs->pgi strain was changed to the BL21/pRSF-NAMPT CP/pCDF-prs->pgi/pACYC-niaP BC strain. The collected liquid was subjected to HPLC analysis, which showed that the amount of NMN was 0.31 g/L.

### Example 9 (NMN Production Using the BL21/pRSF-NAMPT CP/pCDF-prs->pgi/pACYC-niaX SPT Strain)

The reaction procedure was carried out in the same manner as in Example 7 except that the BL21/pRSF-NAMPT CP/pCDF-prs->pgi strain was changed to the BL21/pRSF-NAMPT CP/pCDF-prs->pgi/pACYC-niaX SPT strain. The collected liquid was subjected to HPLC analysis, which showed that the amount of NMN was 0.33 g/L.

### Example 10 (NMN Production Using the BL21/pRSF-NAMPT CP/pCDF-prs->pgi/pACYC-pnuC BM Strain)

The reaction procedure was carried out in the same manner as in Example 6 except that the collected bacteria was suspended in M9 medium instead of LB medium to achieve an OD₆₀₀ of 10. The collected liquid was subjected to HPLC analysis, which showed that the amount of NMN was 0.12 g/L.

### Comparative Example 1 (NMN Production Using the BL21 (DE3) Strain)

The reaction procedure was carried out in the same manner as in Example 1 except that the BL21/pRSF-NAMPT CP strain was changed to the BL21 (DE3) strain. The collected liquid was subjected to HPLC analysis, which showed that the amount of NMN was below the quantification limit.

### Comparative Example 2 (NMN Production Using the BL21/pRSF-NAMPT HS Strain)

The reaction procedure was carried out in the same manner as in Example 1 except that the BL21/pRSF-NAMPT CP strain was changed to the BL21/pRSF-NAMPT HS strain. The collected liquid was subjected to HPLC analysis, which showed that the amount of NMN was below the quantification limit.

### [V-2. Production of Nicotinamide Mononucleotide (NMN) 2]

### Example 11 (NMN Production Using the BL21/pRSF-NAMPT CP+pnuC BM/pCDF-pgi->prs+niaP BC/pACYC-prs->pgi Strain)

The procedure for collecting the bacteria was carried out in the same manner as in Example 1 except that the BL21/pRSF-NAMPT CP strain was changed to the BL21/pRSF-NAMPT CP+pnuC BM/pCDF-pgi->prs+niaP BC/pACYC-prs->pgi strain. The collected cells were suspended in M9 medium to achieve an OD₆₀₀ of 40, and 10 mL of the suspension was added to a 100-mL conical flask to make, to which 7 g/L of nicotinamide, 21 g/L of D-glucose, and 0.05 mol/L of phosphate buffer (pH 6.2) were added. The reaction was allowed to run at 30° C. and 200 rpm. After 8 hours, the reaction solution was collected, frozen at −30° C., thawed, and centrifuged at 12,000 rpm for 3 minutes to collect the supernatant. The collected liquid was subjected to HPLC analysis, which showed that the amount of NMN was 6.52 g/L.

### Comparative Example 3 (NMN Production Using the BL21/pRSF-NAMPT HS+pnuC BM/pCDF-pgi->prs+niaP BC/pACYC-prs->pgi Strain)

The reaction procedure was carried out in the same manner as in Example 1 except that the BL21/pRSF-NAMPT CP+pnuC BM/pCDF-pgi->prs+niaP BC/pACYC-prs->pgi strain was changed to the BL21/pRSF-NAMPT HS+pnuC BM/pCDF-pgi->prs+niaP BC/pACYC-prs->pgi strain. The collected liquid was subjected to HPLC analysis, which showed that the amount of NMN was 0.04 g/L.

### VI. Purification of Nicotinamide Mononucleotide (NMN) 1

500 mL of pretreated LB medium containing NMN was subjected to membrane concentration by filtering the liquid with a NF membrane (SYNDER, NF-S) for 2 hours with stirring at 400 rpm to achieve a final volume of 50 mL. The resulting concentrate was lyophilized overnight to achieve 6.3 g of NMN-containing crude. The obtained crude was dissolved in 15 mL of miliQ water, and after filtration with a 0.22 μm filter, the filtrate was subjected to preparative HPLC to separate an NMN-containing fraction (purity: 64.56%). The NMN-containing fraction was lyophilized again, and then subjected to preparative HPLC. The resulting high NMN content fraction was adjusted to pH=3 with 1N HCl, and lyophilized to obtain NMN (purity: >99%).

MS(ESI): m/z 335[M+H]^{+ 1}H-NMR (D₂O) δ: 9.49 (1H, s), 9.30 (1H, d, J=6.4 Hz), 9.00 (1H, d, J=7.8 Hz) 8.31 (1H, dd, J=7.8, 6.4), 6.32 (1H, d, J=5.0 Hz), 4.79-4.65 (1H, m), 4.59 (1H, t, J=5.0 Hz), 4.47-4.45 (1H, m) 4.34-4.30 (1H, m), 4.18-4.13 (1H, m).

Preparation Conditions:

### Instrument: Agilent Infinity 1200 Preparative HPLC

Solvent: A=100% H₂O+0.1% CH₃COOH
B=95% MeCN/5% H₂O+0.1% CH₃COOH
   Column: zic-HILIC, 21.2 mm I.D.×150 mm, 5 μm, two columns connected
   Guard column: InertSustain Amide, 7.6 mm I.D.×30 mm
   Column temperature: RT
   Flow rate: 22.0 mL/min
   Detection wavelength: 260, 200 nm (PDA) (UV triggered preparative at 260 nm)
   Gradient conditions:

| Time (min) | B solv. (%) |
|---|---|
| 0.00 | 84.00 |
| 50.0 | 84.00 |
| 50.1 | 45.00 |
| 58.0 | 45.00 |
| 58.1 | 84.00 |
| 65.0 | STOP |
| 8.0 mL | |

Fraction volume:

Analysis Conditions:

### Instrument: Shimadzu IT-TOF/MS

Solvent: A=100% H₂O+0.1% CH₃COOH
B=95% MeCN/5% H₂O+0.1% CH₃COOH
   Column: zic-HILIC, 4.6 mmI.D.×150 mm, 5.0 μm
   Column temperature: 25° C.
   Flow rate: 1.2 mL/min

### Wavelengths: 200 nm, 260 nm (PDA)

Gradient conditions:

| Time (min) | B solv. (%) |
|---|---|
| 0.00 | 90.00 |
| 4.0 | 90.00 |
| 21.0 | 50.00 |
| 25.0 | 50.00 |
| 25.1 | 90.00 |
| 30.0 | STOP |

Neplaizer gas flow rate: 1.5 mL/min.
CDL temperature: 200° C.
Heat block temperature: 200° C.
Detector voltage: 1.65 kV
MS detection range:

| | | |
|---|---|---|
| Event1 | MS | 100 to 600 |
| Event2 | MS/MS | 70 to 500 |

### VII. Reference Evaluations

Evaluation of NAMPT Conversion Efficiency:

The BL21/pRSF-NAMPT CP strain was inoculated into a test tube containing 5 ml of LB medium and incubated at 37° C. with 200 rpm for 12 hours, and the culture was then inoculated into a 500 ml conical flask containing 200 ml of LB medium to achieve an OD₆₀₀ of 0.03, and incubated at 37° C. with 200 rpm. When the OD₆₀₀ reached 0.4, isopropyl-β-thiogalactopyranoside was added to achieve a final concentration of 0.1 mM, and the culture was incubated at 25° C. with 200 rpm for 16 hours. 30 mL of the culture was transferred to a 50 mL conical tube, centrifuged at 3000 g for 5 min, and the bacteria were collected. 1×PBS was added to the tube containing the recovered cells for washing, and centrifuged at 3000 g for 5 minutes to collect the remaining bacteria. This procedure was repeated twice. The collected bacteria were suspended in 15 mL of Cell Lysis Buffer (MBL Co., Ltd.), and the lysate was prepared according to a generally recommended method. The OD₅₉₅ of the lysate was measured using the Protein Assay Bradford reagent (Wako Pure Chemical Co., Ltd.), and the lysate solution was diluted with water to achieve an OD₅₉₅ of 0.1. This diluted solution was used as NAMPT solution, and the NAMPT conversion efficiency was measured according to the One-Step Assay Method of CycLexR NAMPT Colorimetric Assay Kit Ver. 2 (MBL). A SpectraMaxR iD3 multimode microplate reader (Molecular Devices) was used for the measurement. The result showed that the NAMPT conversion efficiency was 230.

The NAMPT conversion efficiency was also measured in the same manner as mentioned above except that the BL21/pRSF-NAMPT CP strain was changed to the BL21/pRSF-NAMPT SSC strain. The result showed that the NAMPT conversion efficiency was 170.

As a comparison, the NAMPT conversion efficiency was measured in the same manner as mentioned above except that the BL21/pRSF-NAMPT CP strain was changed to the BL21 (DE3) strain. The result showed that the NAMPT conversion efficiency was 9.

The NAMPT conversion efficiency of human NAMPT (from CycLexR NAMPT Colorimetric Assay Kit Ver. 2 (MBL)) was also measured by diluting the human NAMPT with water to an OD₅₉₅ of 0.1, and the resulting diluted solution was used as NAMPT solution to measure NAMPT conversion efficiency according to the One-Step Assay Method of CycLexR NAMPT Colorimetric Assay Kit Ver. 2 (MBL). The result showed that the NAMPT conversion efficiency was 22.

Evaluation of Nicotinamide Uptake Efficiency by niaP:

The BL21/pRSF-NAMPT CP/pCDF-prs->pgi/pACYC-niaP BC strain was inoculated into a test tube containing 5 mL of LB medium, and incubated at 37° C. with 200 rpm for 12 hours. When the OD₆₀₀ reached 0.4, isopropyl-β-thiogalactopyranoside was added to achieve a final concentration of 0.1 mM, and incubated at 25° C. with 200 rpm for 16 hours. The culture was then transferred to a 50-mL conical tube and centrifuged at 3000 g for 5 minutes to collect the bacteria. 1×PBS was added to the tube containing the recovered cells for washing, and centrifuged at 3000 g for 5 minutes to collect the remaining bacteria. This procedure was repeated twice. The collected bacteria were suspended in LB medium to achieve an OD₆₀₀ of 10, and 10 mL of the suspension was transferred to a 100-mL conical flask, to which 1 g/L of nicotinamide, 0.4 g/L of D-glucose, and 0.005 mol/L of phosphate buffer (pH 6.2) were added. The reaction was allowed to run at 30° C. with 200 rpm. The reaction solution was collected after 1 hour and 2 hours of reaction, and the collected solutions were frozen at −30° C., thawed, and centrifuged at 12,000 rpm for 3 minutes to collect the supernatant. These collected liquids were analyzed by HPLC to quantify the amount of NMN. The result showed that the nicotinamide uptake efficiency of niaP was 81%.

The evaluation procedure was carried out in the same manner except that the BL21/pRSF-NAMPT CP/pCDF-prs->pgi strain was used. The result showed that the nicotinamide uptake efficiency of niaP was 66%.

Evaluation of Nicotinamide Mononucleotide Excretion Efficiency by pnuC:

The BL21/pRSF-NAMPT CP/pCDF-prs->pgi/pACYC-pnuC BM strain was inoculated into a test tube containing 5 mL of LB medium and incubated at 37° C. with 200 rpm for 12 hours. The culture was inoculated into a 500 ml conical flask containing 200 ml of LB medium to achieve an OD₆₀₀ of 0.03, and incubated at 37° C. at 200 rpm. When the OD₆₀₀ reached 0.4, isopropyl-β-thiogalactopyranoside was added to achieve a final concentration of 0.1 mM, and incubation was continued at 25° C. with 200 rpm for 16 hours. The culture was then transferred to a 50-mL conical tube and centrifuged at 3000 g for 5 minutes to collect the bacteria. 1×PBS was added to the tube containing the recovered cells for washing, and centrifuged at 3000 g for 5 minutes to collect the remaining bacteria. This procedure was repeated twice. The collected bacteria were suspended in LB medium to achieve an OD₆₀₀ of 10, and 10 mL of the suspension was transferred to a 100-mL conical flask, to which 1 g/L of nicotinamide, 0.4 g/L of D-glucose, and 0.005 mol/L of phosphate buffer (pH 6.2) were added. The reaction was allowed to run at 30° C. with 200 rpm. Two hours after the reaction, two samples of the reaction solution were collected. One was frozen at −30° C., thawed, and centrifuged at 12,000 rpm for 3 minutes to collect the supernatant. The other was not frozen but was directly subjected to centrifugation at 12,000 rpm for 3 minutes to collect the supernatant. These collected liquids were analyzed by HPLC to quantify the amount of NMN. The results showed that the nicotinamide mononucleotide excretion efficiency by pnuC was 81%.

The evaluation procedure was also carried out in the same manner except that the BL21/pRSF-NAMPT CP/pCDF-prs->pgi strain was used. The results showed that the nicotinamide mononucleotide excretion efficiency by pnuC was 11%.

Production of NAm Derivatives Other than NMN:

A sample of the reaction solution containing NMN obtained after 8 hours of reaction in Example 11 was combined with adenosine triphosphate (ATP) and nicotinamide mononucleotide adenylyltransferase 1 (NMNAT1) (ATP in CycLexR NAMPT Colorimetric Assay Kit Ver. 2 (MBL) was used), and the reaction was allowed to run at 30° C., whereby the formation of NAD⁺ was confirmed. To this sample, alcohol dehydrogenase (ADH) and ethanol (using ADH and ethanol from CycLexR NAMPT Colorimetric Assay Kit Ver. 2 (MBL)) were also added, and the reaction was allowed to run at 30° C., whereby the formation of NADH was confirmed.

Purification of Nicotinamide Mononucleotide (NMN) 2:

The LB medium containing NMN, from which the bacteria was removed via centrifugation, was treated with activated carbon, and the treated solution was separated from the activated carbon. The treated solution was then subjected to NF membrane filtration to removing macromolecular impurities, and the filtrate was treated with ion exchange resin to further remove impurities. The resulting solution was concentrated with an NF membrane, and further centrifuged to produce an NMN-containing concentrate. 5 mol/L aqueous hydrochloric acid was added to the NMN-containing concentrate to adjust the pH to within the range of from 3 to 4, and the recrystallization was caused by adding an appropriate amount of ethanol. The precipitated solid was collected as NMN crystals in high purity (HPLC purity>95%).

### INDUSTRIAL APPLICABILITY

The present invention allows for efficient production of NAm derivatives such as NMN, which is especially useful as various research tools, synthetic intermediates of NAD, and even as pharmaceutical ingredients. Therefore, the present invention has high industrial value.

1. A recombinant microorganism for producing a nicotinamide derivative, wherein the microorganism has been engineered to express a nicotinamide phosphoribosyl transferase (NAMPT), which converts nicotinamide and phosphoribosyl pyrophosphate into nicotinamide mononucleotide, and/or has been transformed with a vector carrying a nucleic acid encoding the amino acid sequence of the NAMPT, wherein the conversion efficiency of the NAMPT from nicotinamide to nicotinamide mononucleotide is five times or more of the conversion efficiency of a human NAMPT.

2. The recombinant microorganism according to claim 1, wherein the NAMPT is composed of a polypeptide with a sequence homology of 80% or more with the amino acid sequence represented by SEQ ID NO:3 or SEQ ID NO:6.

3. The recombinant microorganism according to claim 1, wherein the microorganism has been engineered to express a niacin transporter, which promotes cellular uptake of nicotinic acid and/or nicotinamide, and/or has been transformed with a vector carrying a nucleic acid encoding the amino acid sequence of the niacin transporter, wherein the niacin transporter increases the intracellular uptake efficiency of nicotinic acid and/or nicotinamide by the host microorganism by 1.1 times or more.

4. The recombinant microorganism according to claim 3, wherein the niacin transporter is composed of a polypeptide with a sequence homology of 80% or more with the amino acid sequence represented by SEQ ID NO:9 or SEQ ID NO:12.

5. The recombinant microorganism according to claim 1, wherein the microorganism has been engineered to express a nicotinamide derivative transporter, which promotes extracellular excretion of a nicotinamide derivative and/or has been transformed with a vector carrying a nucleic acid encoding the amino acid sequence of the nicotinamide derivative transporter, wherein the nicotinamide derivative transporter increases extracellular excretion efficiency of the nicotinamide derivative by the host microorganism by 3 times or more.

6. The recombinant microorganism according to claim 5, wherein the nicotinamide derivative transporter is composed of a polypeptide with a sequence homology of 80% or more with the amino acid sequence represented by SEQ ID NO:15.

7. The recombinant microorganism according to claim 1, wherein the microorganism has been engineered to express one or more enzymes which promote a synthetic pathway from glucose-6-phosphoric acid to phosphoribosyl pyrophosphate and/or has been transformed with a vector carrying a nucleic acid encoding the amino acid sequence of the one or more enzymes.

8. The recombinant microorganism according to claim 7, wherein the one or more enzymes are selected from the group consisting of phosphoglucose isomerase, glucose-6-phosphate dehydrogenase, 6-phosphogluconolactonase, 6-phosphogluconate dehydrogenase, ribose-5-phosphate isomerase, and phosphoribosyl pyrophosphate synthase.

9. The recombinant microorganism according to claim 8, wherein
the phosphoglucose isomerase is a polypeptide with a sequence homology of 80% or more with the amino acid sequence represented by SEQ ID NO:18,
the glucose-6-phosphate dehydrogenase is a polypeptide with a sequence homology of 80% or more with the amino acid sequence represented by SEQ ID NO:21,
the 6-phosphogluconolactonase is a polypeptide with a sequence homology of 80% or more with the amino acid sequence represented by SEQ ID NO:24,
the 6-phosphogluconate dehydrogenase is a polypeptide with a sequence homology of 80% or more with the amino acid sequence represented by SEQ ID NO:27,
the ribose-5-phosphate isomerase is a polypeptide with a sequence homology of 80% or more with the amino acid sequence represented by SEQ ID NO:30 or SEQ ID NO:33, and
the phosphoribosyl pyrophosphate synthase is a polypeptide with a sequence homology of 80% or more with the amino acid sequence represented by SEQ ID NO:36.

10. The recombinant microorganism according to claim 1, wherein the nicotinamide derivative is selected from the group consisting of nicotinamide mononucleotide, nicotinamide adenine dinucleotide, nicotinamide riboside, nicotinate mononucleotide, nicotinamide adenine dinucleotide phosphoric acid, and nicotinate adenine dinucleotide.

11. The recombinant microorganism according to claim 1, wherein the microorganism is E. coli or a yeast.

12. A method for producing a nicotinamide derivative, comprising:
providing nicotinamide to a recombinant microorganism according to claim 1; and
recovering the nicotinamide derivative produced by the microorganism.

13. The method according to claim 12, further comprising purifying the recovered nicotinamide derivative.

14. A vector carrying a nucleic acid encoding the amino acid sequence of nicotinamide phosphoribosyl transferase (NAMPT), which converts nicotinamide and phosphoribosyl pyrophosphate into nicotinamide mononucleotide, wherein the nucleic acid comprises a base sequence with a sequence identity of 80% or more with the base sequence represented by SEQ ID NO:2 or SEQ ID NO:5.

15. A vector carrying a nucleic acid encoding the amino acid sequence of a niacin transporter, which promotes cellular uptake of nicotinic acid and/or nicotinamide, wherein the nucleic acid comprises a base sequence with a sequence identity of 80% or more with the base sequence represented by SEQ ID NO:8 or SEQ ID NO:11.

16. A vector carrying a nucleic acid encoding the amino acid sequence of a nicotinamide derivative transporter, which promotes extracellular excretion of a nicotinamide derivative, wherein the nucleic acid comprises a base sequence with a sequence identity of 80% or more with the base sequence represented by SEQ ID NO:14.
